SEQUENCE LISTING (45 sequences)

SEQ ID NO: 1 - DNA, 410 bp, Homo sapiens
  ref <308>: DATABASE ACCESSION NUMBER: NM_199234.1
  ref <309>: DATABASE ENTRY DATE: 2010-03-10
  ref <313>: RELEVANT RESIDUES IN SEQ ID NO: (1)..(410)
atgaagttatgggatgtcgtggctgtctgcctggtgctgctccacaccgcgtccgccttcccgctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 2 - DNA, 237 bp, Homo sapiens
tcgataagccacgccattgggtttgggggactcagcgcacagctgactactgcaggactactactgtggttatgaccatagtaagtttcaagcgcaaagttactagagggataattctgggggtggcgttagggagggaagagtagccagggtggtatcctagttacacacagctcattcctgctttattttggggttgtgtgaaattttctattgctatcttttggccttatcgag

SEQ ID NO: 3 - DNA, 1246 bp, Homo sapiens
  misc_feature (703)..(703): n is a, c, g, or t
  misc_feature (709)..(709): n is a, c, g, or t
  misc_feature (943)..(943): n is a, c, g, or t
  misc_feature (1152)..(1152): n is a, c, g, or t
ctcccgccgccgccgccgccaacagggcgagggctgccggcaactctcccgccgggcccccgcacccccagaagccgaggtccgagcagccgccgctgctttgggtggggggctgacagggctgcgcgcgtcgcgctcttggctggggctgcgcgggcccggggcgctgcgggcggctcagcggcagctgccgcgctctgcgcctcctctgggcgcactgcctgggagcacgagactggtttgtctgatgctgctgccggagctgaggtcttgcctggagatccgaacgagacaccacgtcaaccggcgcggggagtcccgtgaagacatgagggcgccaggagcgcaggctggtcttctagagcccgggctgggggtccggggtccggcgtgggggaggggcagcgcggggccccgacacgtatgggaaggcaaggcgacactcttttccgctcgatgcatatccatcgtacactcccacatctctcccctaagcctccccctgctccgcaccttccaccccttgtcctggcacccccaccacttctatcctaaccttgcccagctccctcccactcatccaggtagccggctgtcgtgtttcgccaccactcccctcccttcctgccggtaatcggtcgggacccccgggggggcacggggcgcccccaaaaaaaacaacaaccagaaaaaaagggggatntttcgntcccccgttccgcctccttgctgcttactgtccactacaatgccttggccttctccaatccaattcctcccatcccatccccgcaaccagttctttctcccccgcttcatttccttgttttatcgcatatgtcgtccctcctactatcgtctacccccccattgcacctcatggtcctccccgcccccgatgtacttaattgacaatttgccccgcacataccttttaccctcnacacacaccttacgcgcagtgacccacgtttactacactacccactttattccccaccggtttggacgttcccattccgtgggcttccttgcactttaccacatatccaccccacgcatatgctatatcccagtaacctgccaattactcccgcctggtaaatcatacccccccttattccccgtcacactatcatatcccacttccanacccacctataaaccacatcaagaactatctcacatatctagaaatcttttccaatatttgccctgcctcaaatcgatgtacatcctttgacat

SEQ ID NO: 4 - DNA, 684 bp, Homo sapiens
  misc_feature (366)..(367): n is a, c, g, or t
  misc_feature (628)..(628): n is a, c, g, or t
  misc_feature (647)..(647): n is a, c, g, or t
  misc_feature (652)..(652): n is a, c, g, or t
  misc_feature (662)..(662): n is a, c, g, or t
atttgggctttttctctaagagtaacagagagctattggaaggttttgagcagaggaggttatgttctgcgtttaaagtatccctctgggtgctgggtagaaaatagatcgaaggtgggtaagggtagaatgaggaagcccagttcaggggccactgtggttatccaggcaagaggtgacagggcttcagccagtgtggaaacagctttgtgatttccgtgcagctgatcctgccaatgctaaatttcagatagcaactaggcattctacggattccaatcaccagtcttttcattattatatatcatatgttttatatattacacacacacacacacacacacacactctacacacacgtggttnngttgcttttgctagattttgttctataggagggatacctgatatattttaacaattaaataggttaggtttatgacaaaacaatttgacatgggttagacataatgtaatctttctatgctttaatacaatcttggcaaaaaacatttactcttgctgacaactcttctttggtttatttctctaagccacttagaatcctaaacatactaaatgacctgaaaaatgaggtgaggggggcataagaggcctactacccagnaaacaggtgtttggattgnttttnccccccgttnagaaatttattacattgttcca

SEQ ID NO: 5 - DNA, 20 bp, Artificial Sequence
  Antisense oligonucleotide
caccctggctactcttccct

SEQ ID NO: 6 - DNA, 19 bp, Artificial Sequence
  Antisense oligonucleotide
ggctactcttccctcccta

SEQ ID NO: 7 - DNA, 21 bp, Artificial Sequence
  Antisense oligonucleotide
tgtgtgtgtgtgtgtgtgtgt

SEQ ID NO: 8 - DNA, 20 bp, Artificial Sequence
  Antisense oligonucleotide
ttctacccttacccaccttc

SEQ ID NO: 9 - DNA, 20 bp, Artificial Sequence
  Antisense oligonucleotide
gtcgccttgccttcccatac

SEQ ID NO: 10 - DNA, 20 bp, Artificial Sequence
  Antisense oligonucleotide
  misc_feature (8)..(8): n is a, c, g, or t
ggtgggtntggaagtgggat

SEQ ID NO: 11 - DNA, 13 bp, Artificial Sequence
  Antisense oligonucleotide
cggcagccctcgc

SEQ ID NO: 12 - DNA, 14 bp, Artificial Sequence
  Antisense oligonucleotide
tgggggtgcggggg

SEQ ID NO: 13 - DNA, 14 bp, Artificial Sequence
  Antisense oligonucleotide
ggacctcggcttct

SEQ ID NO: 14 - DNA, 14 bp, Artificial Sequence
  Antisense oligonucleotide
gcggcggctgctcg

SEQ ID NO: 15 - DNA, 14 bp, Artificial Sequence
  Antisense oligonucleotide
ccacccaaagcagc

SEQ ID NO: 16 - DNA, 14 bp, Artificial Sequence
  Antisense oligonucleotide
ccccccacccaaag

SEQ ID NO: 17 - DNA, 14 bp, Artificial Sequence
  Antisense oligonucleotide
gcgcagccctgtca

SEQ ID NO: 18 - DNA, 14 bp, Artificial Sequence
  Antisense oligonucleotide
cgcgcgcagccctg

SEQ ID NO: 19 - DNA, 14 bp, Artificial Sequence
  Antisense oligonucleotide
cagccaagagcgcg

SEQ ID NO: 20 - DNA, 14 bp, Artificial Sequence
  Antisense oligonucleotide
ggcccgcgcagccc

SEQ ID NO: 21 - DNA, 15 bp, Artificial Sequence
  Antisense oligonucleotide
gcccgcagcgccccg

SEQ ID NO: 22 - DNA, 14 bp, Artificial Sequence
  Antisense oligonucleotide
gaggcgcagagcgc

SEQ ID NO: 23 - DNA, 14 bp, Artificial Sequence
  Antisense oligonucleotide
cagtgcgcccagag

SEQ ID NO: 24 - DNA, 14 bp, Artificial Sequence
  Antisense oligonucleotide
gtgctcccaggcag

SEQ ID NO: 25 - DNA, 14 bp, Artificial Sequence
  Antisense oligonucleotide
ctgcctgggagcac

SEQ ID NO: 26 - DNA, 14 bp, Artificial Sequence
  Antisense oligonucleotide
aagacctcagctcc

SEQ ID NO: 27 - DNA, 15 bp, Artificial Sequence
  Antisense oligonucleotide
ttcggatctccaggc

SEQ ID NO: 28 - DNA, 14 bp, Artificial Sequence
  Antisense oligonucleotide
tgacgtggtgtctc

SEQ ID NO: 29 - DNA, 14 bp, Artificial Sequence
  Antisense oligonucleotide
ctccccgcgccggt

SEQ ID NO: 30 - DNA, 14 bp, Artificial Sequence
  Antisense oligonucleotide
atgtcttcacggga

SEQ ID NO: 31 - DNA, 14 bp, Artificial Sequence
  Antisense oligonucleotide
ctcctggcgccctc

SEQ ID NO: 32 - DNA, 14 bp, Artificial Sequence
  Antisense oligonucleotide
aagaccagcctgcg

SEQ ID NO: 33 - DNA, 14 bp, Artificial Sequence
  Antisense oligonucleotide
gctctagaagacca

SEQ ID NO: 34 - DNA, 12 bp, Artificial Sequence
  Antisense oligonucleotide
cctcccccacgc

SEQ ID NO: 35 - DNA, 25 bp, Artificial Sequence
  Antisense oligonucleotide
gcaggactactactgtggttatgac

SEQ ID NO: 36 - DNA, 23 bp, Artificial Sequence
  Antisense oligonucleotide
ccacccccagaattatccctcta

SEQ ID NO: 37 - DNA, 16 bp, Artificial Sequence
  Antisense oligonucleotide
tcaagcgcaaagttac

SEQ ID NO: 38 - DNA, 17 bp, Artificial Sequence
  Antisense oligonucleotide
gccggctgtcgtgtttc

SEQ ID NO: 39 - DNA, 17 bp, Artificial Sequence
  Antisense oligonucleotide
agcaaggaggcggaacg

SEQ ID NO: 40 - DNA, 16 bp, Artificial Sequence
  Antisense oligonucleotide
cttcctgccggtaatc

SEQ ID NO: 41 - DNA, 25 bp, Artificial Sequence
  Antisense oligonucleotide
catgttgcagacccatcgcctttga

SEQ ID NO: 42 - DNA, 400 bp, Homo sapiens
  misc_feature (19)..(19): n is a, c, g, or t
  misc_feature (21)..(21): n is a, c, g, or t
  misc_feature (34)..(34): n is a, c, g, or t
  misc_feature (39)..(39): n is a, c, g, or t
gaacaggtttttttgaaananaggattaaccttnttccntaacttcctttagaagtcttaatttgccttataaaaaaatccactttctatctttcttgggacttgtggaagagggtgcttttgttgctataattaagcataaaataagcacattgcatttactgggttgtttccttcgataagccaaggccattgggtttgggggactcagcgcacagctgactactgcaggactactactgtggttatgaccatagtaagtttcaagcgcaaagttactagagggataattctgggggtggcgttagggagggaagagtagccagggtggtatcctagttacacacagctcattcctgctttattttggggttgtgtgaaattttctattgctatcttt

SEQ ID NO: 43 - DNA, 619 bp, Homo sapiens
tttttttggcaacgtctgggttagagaactattatctgccactgtgttcctgtccctcttgtctagaccgtttgagtgaatgaatagccagttttggaagacccggcactaaggggcattttaaagtttctgttggagaaacgtttatatacattaaatttgtgatggaaataaaagcctttaaagtaggtttatacagttaattgacagacactcttggaacaggtactttagtttcaaaggattaaccatgttccctaacttccttttgaagtcttaatttgccttataaaaaaatccactttctatctttcttgggacttgtggaagaaggtgcttttgttgctataattaagcataaaataagcacattgcatttactgggttgtttccttcgataagccaaggccattgggtttgggggactcagcgcacagctgactactgcaggactactactgtggttatgaccatagtaagtttcaagcgcaaagttactagagggataattctgggggtggcgttagggagggaagagtagccagggtggtatcctagttacacacagctcattcctgctttattttggggttgtgtgaaattttctattgctatcttt

SEQ ID NO: 44 - DNA, 813 bp, Homo sapiens
ttgtggagtagaaaggcttctctttatggtacagttcattgagctctaaagtaacttgcctgaaagtcacagagttagttgaaagcagagccaggagcctgagccagaattaaatgaatctagttgaacagggcactaatacagcaattaaacctccatgaattaatcagctttgtaatcaggcacagtcatttttctcttggcaacgtctgggttagagaactattatctgccactgtgttcctgtccctcttgtctagaccgtttgagtgaatgaatagccagttttggaagacccggcactaaggggcattttaaagtttctgttggagaaacgtttatatacactaaatttgtgatggaaataaaagcctttaaagtaggtttatacagttaattgacagacactcttggaacaggtactttagtttcaaaggattaaccatgttccctaacttccttttgaagtcttaatttgccttataaaaaaatccactttctatctttcttgggacttgtggaagaaggtgcttttgttgctataattaagcataaaataagcacattgcatttactgggttgtttccttcgataagccaaggccattgggtttgggggactcagcgcacagctgactactgcaggactactactgtggttatgaccatagtaagtttcaagcgcaaagttactagagggataattctgggggtggcgttagggagggaagagtagccagggtggtatcctagttacacacagctcattcctgctttattttggggttgtgtgaaattttctattgctatcttt

SEQ ID NO: 45 - DNA, 19146 bp, Homo sapiens
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactgtaagaaccgttccctccccgcgggggggccgccggcggaccccctcgcacccccacccgcagccagccccgcacgtaccccaagccagcctgatggctgtgtggcctaccgacccgtgggcaaggggtgcgggtgctgaagcccccaggggtgcctggctgcccactgctgcccgcacgcctggcctgaaagtgacacgcgctggtttgcccagcacagaggggatggaatttttatgctgctcctttagcattctgatgaacaaatatcctccccaccagcaccaccacctcagaaacacacacacagctgtccccttttctgtttcctcacctatacacactcccagtttttctttgcttccaaagccctttatctgtgtgtctgtgcctggctgtgcttaattctgagaactattgcactttcatcctaaactgcgcctgcaaggcgagaggccggcttttcacaaaagcaagccagaggcagagaaaacacagaagggcctccatttccagaacaagcgtctgggtaatgtcaaatctgttcagaaaagttcctctgttcagaaaagttccggttctagaaagacttaaccataaatagtgctggctgggactagggacaaagactgtagctcactccacgtgagacaatgctaactcttgagaaaaaaacccaggttattcttattagaaaataagtctgtgatttacctctcaaaaattaacatgttttagaagcaagtcaattagggcatatcagctgtgatgtgatctcgttttccctccactcctcagaagcttgttgcatgaagtgagagaggctacattacatgtgatagaggctgttgcagaggcataatgtcaacaaagatagcaatagaaaatttcacacaaccccaaaataaagcaggaatgagctgtgtgtaactaggataccaccctggctactcttccctccctaacgccacccccagaattatccctctagtaactttgcgcttgaaacttactatggtcataaccacagtagtagtcctgcagtagtcagctgtgcgctgagtcccccaaacccaatggccttggctaggaaacaacccagtaaatgcaatgtgcttattttatgcttaattatagcaacaaaagcaccttcttccacaagtcccaagaaagatagaaagtggatttttttataaggcaaattaagacttcaaaaggaagttagggaacatggttaatcctttgaaactaaagtacctgttccaagagtgtctgtcaattaactgtataaacctactttaaaggcttttatttccatcacaaatttaatgtatataaacgtttctccaacagaaactttaaaatgccccttagtgccgggtcttccaaaactggctattcattcactcaaacggtctagacaagagggacaggaacacagtggcagataatagttctctaacccagacgttgccaagagaaaaatgactgtgcctgattacaaagctgattaattcatggaggtttaattgctgtattagtgccctgttcaactagattcatttaattctggctcaggctcctggctctgctttcaactaactctgtgactttcaggcaagttactttagagctcaatgaactgtaccataaagagaagcctttctactccacaatcccagtggctatgagatttgccccttggcctttatgtttgtcctaaagtcctttgggaagaatctcctaagaagagattggagtcacctggggaactttaaagactactgatgctaggtctcaccccaggagattctgatttagttggtttggggtgtggcccaggcatctgagtttttaggtgttccccagatgagtctaatgtgcaaccagacttgaatcaccatggcttgataacacccaagaaaatctttggctcatataaggtacaagtagtcacaaagcaagcgagttaacacagattgcagggacaagggcatgttgtagggaagagggattgcttcccttttccaaaattgatgctgtgtcatcagtgaacaagattctcactactctatttacatttgaacagcaaaacacaccccattgtgttttgtctgtgggaagacccaatagatcccagaggaaacctgaaaaagaaacgttcccaaagagaaactgagctgcattctaagccaaattgacttctttccagatatgtttgattcggtagcaagctgtcaagtgcagggaaggcaaaataatgacagtatgcagactttgaacactcaaccagtgtcaacacgcctctgtcacagtgctgacatttatatcctgcactgtacatggtgcaactggttaaggcttatgtagaaaaacattaagtcaccacatttcatttaaaaatagaaagtatcataaattctgattcctctagttccatccaaatacttgtaacttaatgattgagcaaaagagctttcatgccacattaacgtcattctgcgcttttcacaggaggaccagaatatacacagtttgcacactcacctttaagattgcatgtgttccgctggttccaatgtaaataaaacatccagaattctattactagtatgccctcagtgtgagataaccaaatggaattcataattggccaacgggcttgcctagaggctggctgtaaagtaaatggctcaggactgcctcctgtagcaacttctagcctgtctaaactcaaggacttgtgatttgacttttggggtacccaagacttttctcttttgctatctttatttgtttattttgctttttgccagtattttgccagtattttgctttttgccagtttccaaagggcaattagagcaggcatgaggaatctgcaagttgaaacttgcaaatgtcatagcattttcgagctgaaaggaaacccctctagtcctgaaacctaaagaggcaaagcaacttgtctataatgcaccacagttccatgatcacaggaggcaggtctgaagctgggaccagagtttcccccaaactactgaaatgtactttatatgggaaggggaggatactttatatgggaggaaggtcttgaactaatgatttagaccatggtgcacaaggttgtctgaatttattgaaggttagttcatcatagtcatattccttatctcaactctaaatgtatttaataagtaaaagcataaaatgcatgtggttttaaaaatttgcatctaaatgcacatatacattcaccaaatagttacatgtatgcctgtctgtgccaggcactgtcctaagtgctagggataccttgatacacctaataaacaaaagtcgctgccctcatggaacatagcttctagtaaatgcacatgtgtggtggtatgaaaaaatcaacgtgaaagaacctcagtccagtaaggtgggctgaattttttgtgcaactggaaaccaactaaaatgacattgagcatcgttaagttgtaattacaagtaattcctaatttagtataatataatgggattatattctaccagtttggtagcagttcacaaaatttcctatggaaacaattttttaaattaagctatcaagttttcaggctagcccacaaaaacctatgtaaaccacgatataactgaactgtcattcacaatattactaagtgagagtctctgccctggggggcacatcccataatctgcaaaaggagggcacagaacaagaaatcagggaggtttcagcaagcaagagctcaaggtaacacattcagaccagactaggatttggaccccagccttgccacttaactgagatgttgagctgttacttaacatcccttaagctttgttttttcttccctgtaaaataggcataagagtaaatctgtctcatgggttgatgtgagggttaacttagatattgctttgtaaagcctctgctccataaatagggatcatagttgtcatttgccaccctcctgcttgcaatggcccctccctgggcacccagtgaattcacttcttccgctacgacatctgccaggatgctgctctaaatagtgggatttcagcagcacaacagagtacagcgagtgagagaaatgcaggccttgtgggcaggctattttgggccccactgttggttggtcagcacgtaggcagtccaggcttgcctgggattctgcccctgggtcagctgcccctggctcttcccaggtttcacagctccgacccttccccagtttccaagcccatatatttccagtggaatttttttctcaccaaactcctatggttatgttagggagacctcttccgtcatgggaaggagggaaccaaggcagggtaggatctttctatggaagtgacaagagggttgtgagttggaggctacctgtatggagatggagcgtatctttgtaaatgatccatcccagctggacatttaatttaagaagtttcataaccctagcaaaaaatacttagtaaactgtgagaccacctctatagagctaaggaagcttctagtgcaacaagctgggtaaatgttaagctttcaaatctaatgttatgaaatacagaatttagaatgaacaaacaccatctatctgttgtgtcttatcgcttagggactgtatgaatgaacaatactttaacaaagtaactacctaagctggaaaggagctctgccacttgggatgggtctcttggaatcaagaaactgcacatcagactcttgtaggaggcactgattctttacttgagatctgagaccaatattctcattctggccccagtctggaaaaccatttcaaactgttactgcttagagaaatctatttaaggcttaaattgatttggccagtcaggaccttaggctgacccagggcgtgagcaatgtactcaacaggttcttcttcaacagatataaaaattaaagctccctgatattttcccctcctaatatctctttgatctttgtttatttaacactcatggaccatagcaccttcatttgattaaaatacttgttttttagttaattacttaagcttaatcaggaaacaaatattaaatatctttcatgtactgggtgccatgcggcctcaaaaaggctaaatttgtaaataaagttgcttgtgatctagctgtaaactattgctaggtggatgggcggatggctggatggatgaatagaatgtcagtcagatagatatgtctattgtgtagggtttaggtgagctgtacgcatgtgtggtaaaaattcctagcagtgagctcactgggatttgggtatcatagcagggacgacttcagggaagagggaggatttgacctggtacttgaaagatgggtgtttggaagggaagggaaaaggaaagggggacatactagggagtagcatgggaaaatttgtgaccatgatgtggggaagggacagtggaacaaagaggaggacagaccagtctccaagttctagaaagaggtgctaagaagtggtgggaaattgaagatgtcttgacaggtgataccagattgtgtgatactataaaattcaggaaaggtgtttgctctgtttttgatagcagtggggcacccatggagcaggcaggtgactacaaggcgggatcttgcttcaaagctgtcttgcaggagaagttacctaaatacccagactgcttttgagtggggcctatgccgtaagcatgtcttttattttgaggtgttgtgcttttatttttaaaaatcttctagacataggcacaattgaaccaaactattagccccaagtaagtgaatatctgcaaataaacacttaatattaaagttaatgacctagcaccttttaactatccacatgatgaacacagttcttgtcctgaaatcaagtaagtctgagctctacaatagaagaggagatattattatccccattttacaggtgaagaaaggagacctagagagttgttccgggtcacaaacctagtaagtggtgaagccagaatttaaacctcagcatgttggtccgaaagccaatatttcttgactttacacagactgtgtgtgcatattagtgaattaagaaaatatagactttggcttgcttaaaaatgcactcactaaccctgaaacacagatttccaggaaaattaagcaagcaaagagaaaagagaagcagagactatcaaatttccttttggcccttttaaaatctccatttgggctgcggtaggcttaagccagtattactaatgactacttcaaagtccagatcaggatgtttttaagaagagaaacatgaatttctctaagtattcctataatattgatgctttttgcaatgagagaaggctccctaactctttgcaacaaagcaaggtctcctcaagtgcttggtaggcagacagcattcgggaggccttgtgggagactctggttctcagatctctcccattctgcccagcgaggggatggcatccacataggaacctagtgtgactgcacgagtgccagagcgatggcctcagtgggaataggagtatgcgtaagcagacttgggtcaccactgggaaacaactggttagctcagtagaggcaaaacccacttttgcataactttaataacaaaattgaagtagagaagcatggttttaaaacaatatggccttccaatttttttgcctgcaaacctgcaaataagaatgttgaaaaacgattacctactttgaagctttctaaaatttttcatcataggtttaaatagttacaccagatgtcatttctagccctttcaggaactgtatatatgctttaaatattcttttggcaaaactttgcacctgcctatgtagtttatttagttcatggaaaatgatcaaactaatcagcccaaatcaattggctttttggtcagaaaaggtctgacttcattttcaattcaaatgtacatgttaatatacaccccatgacaactgtcacaattctggatcctaattgtaaggtagagagatggataggtgaacagtcagataagcaaagaaagcactgaggcttcccaaggggcctggccaggaggaaatatggagcaactggcttcaggatttcttggtttattcacaaagttatctccctttgcaagtgtttgtagcacagtgaacacccatacatccttcactaagattaacctcttttttagcattttgcctcgtttttttctgaaccatttgagaatttgttgcaaacatcagaatgttaaatatttcagtatgtaccttctaagaacaaggacaatgtcttatatagccacaattcagttatcagtctcaggaaatttaacatggatataatactgttatccaatagatagtctgtgttcatatgtccccagttgtagcaattatgtcctttatgttttgtttttttttttttaaaacccaggaccataaattgcatttgactgtctggtatctttatacttccttaatctaaaatgattcccaagtctttttttcccgcatgacattgacatttgtgaaaactctgggctcttttgctagctgtttcccagactacccttcagtttagatctgtctgattatttccacatgattagatgtagggaaacatccttggaatatcatattcatggcactatgtcctcacatcaggaagcgcccaatatcagtttgttccaaggagccattcatgcatgaatgcctttgtatggcgcctgaaggtgtttatccctgggacaggccccacaatagaatggctagataggtgagaagcacaccgtctctcagtaaggataaagagggattggaaaaggggacatgggcaaggagaaccacagcaggagcatccgcaggcagaccagacattttaggacatggcacatgtggaaattgaggaaatggacatggattccctttcaaccacccataggcactgcagaaagtcttcctgtgcccccagagggccaggtgcctcagtctgaagacactgctctaagccatagtggacacaaagataaccctcatgggctctggggtctgctgacatttgcatgtttagttgtcaggcattttgaggaacacggaagagctcacacagcctcccaagacgggcctgtggcttctgcccacaacccaccatgccaggggctccaggccctgcacagaggtctctcctctgtagaacatgctagactaagtagggaccagtgtctggatccccttttgggcaatcttgctttgtctagagactgtagagttggctttacactgctgccttctgtgcaaatattgctgaaggaggatcccagatgtgataaccatattggcctacattaggcaccatttgtgaaatactttctgtggaccaggaactgagctgaaatcttttcttatgttatcatattaattttgacctgaaggtaaaggtgtgactagccccatttgaaagatggggaaattgaggcttacagggttagcagcttagccagggtctggacacagggcagcctgagtccagcactcacacttgtcactactgcacaagatcacttcctgccagcccatgacactcagatgtcactcttctgactgcatcaaaaagtcatcaaggaaaaaaaatcagggaatgggtttggcaggtaaagttgttttagaatgataaccgtatctcattacctgaagagtctttgagattcccgtaaattcactcactgggggtagaaattccctcctcatatctgaccacaagaatctaccaaacaaattcaaatgataaagaagatttctttcatcttctcttagtccctccttcttgttcaaatcactgagacccttacatgccttttacctactgctcagtgggtcccctgggaagagctgagggatgctgagtgcagaatcctgcagggtcctgcagcctctcaggctgggggcagggcttcgctgaaagaagaaaggagagactccaccacccccacaaccacctgcctctgataacaccacagggatgatttgccctagtggctttttgtgcacttaattttactgggtaactttctcacccttccctgttaagttttttataagggcagtagcagacctcctggtgtgtgctcattagcttgtggtgtttattaccgactctggagtgcttttagctcacaccaggtgttcatggattagctaacaaatgaacaccctgttgggtgttttcctgacttaaaagctgaagaatggacactttccacccagggggactgtgctgtattactgtaaaattattagcataactgtaataaaagcatggaccacatacatagaaatcagagcaaggtgatcagaacctgagtaacaaaggaatttactgtctgtctctccctgagtggggttttctggctgtttgtattgatggagtaattttcagtccatttattacaaatttgcttagttgagttgtaggataacagtttaggatatagtacaactagtatgtgcaatgtcattcagagtgggtggagatggtaaataagatggcattttgatgggcaaagtggcttttctaagtacacccatagcttctttttctatattctaaagtgatttgcattctggttggtctttttcttctgccttgagagaatccagaaatgcttttttaaaacaacaaaacagtggtgtttttacaacgcaaatacttttcaaataaatcgatgtcatgccttactgtcaacaaaccactggtcctgaagagaatgcactggtagctctggaaatggtcacaatgacttagtaaattgcctcagctggtaattgtttttaggaaaatagatgctgtggacacttctgaaagttaacccaacagcagcctatgatcaggacggtctaccaaacactagatgaattctgtgtccaaaataggaaagcacggaaggtcattacataatgtaagatgcatcagcattcagtgcttactgatctatgggccttttttaaaaagtagttcaaataagtgtcaaagtcatcactttgaaataggagcagataacaaaactttacagaagtttttcagagaactagaacattctcataaactcacatttagagtccattctcatggactgcacattttagaggttcctgaaggtcaaataagaacaagagttgacagcccagagattggcttcaaggacaagctgcttggctctcctgatccattttgtaccacttgcagtgggcaattctagccttggagtcataagctgggtatgacctaagcatacttgaagcagcaaaaacagaaatgaataaaattgagattcgaagaaaatggtaaattgagtgtttgacttttgggtgatggagactgaaggaattgtagcgtgaggctgtagtgtgtcctctcaggggtcatggggcccatctctatttttacagatggaaactgaagtttaagaatgccttacagtagaatctggatgctttcatatgcagatacagggccctttctacacatcttttacctctcttaaatagggcacaggaagatgacttgatgatttaagagaagattgatgacggtcatttcaaatgtagccgagacattcagccaagaggtaaactgaagaggtcaagcactgcagagttctaaaatacctcctgtggggttttatggggcccctgatggtactggctgagctgaatgctgctggggcgtcagccagaggtggtctactcccttgcagaggtgactgaaaaacccgtgtctggccacactttccagccaagacttagactctccacactttagcattttggagctggaaaaggccccagagagcactgagttgacctcagaagggttaagtgactacttccaaggtcacgcagctgatcagggactgacccaagactggaatccgggcctctcttgtctccaactctgcagcaagagcctggtcatttggtgccagcatgagttggaggagcttccggagatggggcctctctgtctggatctgctgctgtgctggctgcggcttttccggttttaactgggaaatcgccagagctgtcttagcgtgatatgcaagaaccaggacacaggagaaatgcccctgagtagcatggcttttcctttttgggagacaatttactgtattctgtggcccatggcagcctaactttaggatctacttagcgtacctgagttcgtactgaatttttcaacagaaagtatgtttctcacctcctgtgctgactttggtaaatgtgtacaggtgaaaccagcatgtcttgctctcgtctcagagtaaattcccatctgctgcaagacttgaagagctcagtggtagtagagtttttactttaaaacaaaaagacaacaagtttgagctttgaaactgaaccaccaggtccacatttattagtcctatggacttcaccaaatatttgtcttctctgagcctcagcttcctgagctgtaaaatggagatagtattcaatttagccttgaagggaggttgtcaggtttaaagttaatgttgtgtgaaagtaggcagtattccacctttacactggtaatgttttttaagtgcttcaaggaagctcttatctaagttatagcctgatgaaattttgtaatgcaagaagttttacaggttaaactcagccatgtagttcttgtaatgatattccaatattagtgaaagaaaatcatgtttgtaacatatagaaagataaaaataacttattccaaaacaatgacattcattgggactcttcttgagaagttgtatcaattttaagttgatcttttcttttcatataagtatgtatatgtgtgtctctgtgtgtagacgcatatgtatgcacgcatgtgtatgtgtatgtgtttatagatcgagagagatctctatatttttcagatacttggcctgtagagcaatttgcattttacctgcaatataggtaggcaaagaataccagattaaatgtctcccaccattagggggttttccattttcctcctgtctgaagacagcctttcataggaaccgctacatgtcaggatggatggcaccgcacctcggcagccgcaggtgcagcatcctgtggcctcatcctcagcatcctgccccatcccaaaactgagccccaccatctggccactggttcctgatgtatctctcacttgtgtggccttggctcaggcagcagctcacccctatgggtcccattagccctcacccttccaggcacagaagctggacactatagtgaagtagcaaggctgttctccccacagcaagaaacccctgagccttctctctggggcccctgcatcagccaagcccggctcatgcagatctcaagcctggcctggaagacatctcctgaacaggaccactgtgtgtactgaggtcaggccccctctctcctgggtccctctgagcctcaaagaccttccttccaccttctggataaggagttgtgtcctcaccactttgtccctcatagacaactttgtgccatctccctctcaggcccacaggttggggacttaggatgacttagaatgaaagtcagcttagactccctgcctggccaggctgaggaggggacaaccagctacaagtagaaaaatgaccctttgattaaaatatagttaacctgtactgttttaagacaaatctggcattttacgaaaagactttgtccactcttgccagcttaaccaatgtaatgttttggattatgtaattttgagttcagctattacagggaggacttggagccctcaccattgttcctattaaaactcagttgactttacaaaatagtcactatctcagtcctttgttcctgggatgacattactattttttttccccataatcgagggcagcaaagtctttgcggtgaactgtttgttcccagagttgtgccccggtacaaggtttcatttctttaagtaacactatttatacaaagaacacatcaaagttaaaatattttaaatagaataacaccaaaacatttggttgcactctgaaggatagcttaaatgcatttgagcactttggatttccaaaaactttatattttagatggacatgttttccaagatatatcctccaagcaggtctgggaaatgagcaaagatatgacctattatttttttaatgatgctcctcttttgtggtttgcaagtatttcactattaaagttaataaatgagggtcatttgcatatctgacattctgccaagccttttaaaggatgaagacagagatgcgccagtaagtcttaatttagggaacgaatccaaacccagacccctcttcttttccctcagctcagtgaattcctggaaaatggaggcagctgtgggcatttcagctcatcaccaggagtttcttgtctgggctgggtgagaggcctctgcagaagaattaaggacaggctcagtgaggctgcccagcatcctctgcagaggagtatggcgcctaatgcccccaggtgcctcgcatcgataaattgaggctggctctaagaatgaactcatttagtcggaacatgcaggcctatttgccctgtggtttgaaaaatacaatgttgccctttcctggcttccagaattcatatccatgtaaatttttcagcaaaaattttgttgtttcttctatttttcatgactatgaaggaaaaaaaagccttcagcttaataagagttgcctatggcctgaacttggggtttaaataatatttccacattagcaacaaaatgtgaaggagatttcccatcaaaggaaggaattttcaaaagccacatgccaggaagactttaaaaaataataataataatagactttattttttagagcagttttaggttcacagcaaaattgaaaggaaggcagagagttcccatatactccctacccctacacatgcatagcctcctccattgtcaacatcccccaccagaatgctacatttattatagttgatgaacctacattgacacatcattatcactcaaagtccacagttgacattggggttcactcttggtgctatacatcccatgggtttggacaaatttgctatgacgtgtattcactatttacctgaaattcaaatacaaccaggctttctgtattttacctggcaaacaaagcctgtgacaaagccatgtggtcaaaatgtcttaaaaaggggagaaaaatgtttttcaagttcttccaagtatcaagggctaaaaaagaacactataagtgctgattcaaatccttatgattgtaactctagtaataaaaagttatatatcatatagattaatgagtctctaaaccagcatgatttaaacttctgtgactctaatgttttcctattagcttatattaaaattttagtaatgttttaccctcatttctgattaatcttcagttgattgttaattgaacttaactttctatatgcccaggttcctaaattaatgacttatttagaattcttgaatagagcctatgatgtgagtccttttgtaaaacagagtctctgttttttaatttgaagactcttctgtattttgattaatcactcctaatagatctgtttcataagttccgatttatatacaagcatgtttttttttgaaactgggcacatctgtatttatttcctctttaatagcttattattgaaagaaatcaccaatagctaaagcagctcatttttttttaagtcaattgtttctgtcaaccaaagtgatttaatgttgttgttttgttttgttttgaaagaaaacttcctaaagaaagttcagtgagaaagcagtacaaaggaaaggacaaattaagagcactcttggccaggcacagtggctcactcctgtaatcccagcattttgggaggctgaggcggacggatcacctgagatcaggagttcaagaccagcctggccaacatggtgaaatcccatctctactaaaaatacaaaaattagccgggtgtgatggcgggcacctgtaattgcagctacttgggaggctgaggcaggagaatcacttgaacccgggaattggaggttgcagtgagccgagaccatgccattgcactccagcctgggcgacaagagtgaaactccgtctcaaaaacaaaaaaaaaaaaaaaaaaagaaaagagcacttccctgggcccccttaagaaagcattactcagggaccagagagctttaggagacccaggggaggtgggcactgagagttaatgctgctgcaaagctgcagaaagctgaggaggaaaggtggtttggagacagtccttcagctctctggtgggcaatgactgtgccactcagggcagagtggcttttctgagtaatgtgggctgtttactttggagcatttcatttcataacgatgcagttttcagctaaagtcccagagtccccttgaaaaattaagaactctctttccgcctagtaaaatgacccgactcattcagccagcagttaccgtaggcctccacagcctactcaagcacttggagatccatcagtgaacaagcaggacagagttcctgcctcttggagttgacattcttttagagaagacagactataaataacaaccctaagaaatcagttagatggtgtgttagaatgtagcaagaattatggtggagggggaggcgcagggcaggggtatctggattaggggctggggaggaatcttgttttaaatagggaggtcaggtgggacttgtggatagtgacatttgagcaaagacttgaaggagatgtgggactagccacagggtgggagacatttaatctggtattctttgcctacctatattgcaggtaaaatgcaaattgctctataggccaagggtccaggcagagaggggacagccagcacaaaggccctgagttcttgactgtttaagctatagttctcaagggatggtgttgcccctaggggacatttgcaatatccagagataccttgatctgtcatgactaggatgggggatgctatgggccttgtattagtctgttttcttactgctatgaagaaatacctgagactgggtaatttataaaggaaagaggtttcatggactcacagttccacatggctggagaggcctcacaatcatggcagaaggcaaaggaggagcaaagtcacatcttacatggcggcaggcaagagggcataggcaggcaggggaactgcactttataaaacaatcaggtctcatgagacttattcattattaccagaatagcacaggaaaaacctgcccctgcgattcaattacctctcactgggccccctcccacaacatgtggggattatggcagctaaaattcaagatgatatttgggtggggacacagccataccatatcaggcctctcctggatggaagccagggatgctgccaaacatcctacagtgcacaggacagcctcccacaatgaagaatgacccagccgaaaaggtcagtggtgctgacactgagccccgtgtttgaggaatggtaaggaagccagggtagctagacgggagggaacaagaagagaaacaagagaggaggtcagaggtaataaaggctggaggagaggggcagccaggtcaggtagggcctttagggccagtgtggatttgggctttttctctaagagtaacagagagctattggaaggttttgagcagaggaggttatgttctgcgtttaaagtatccctctgggtgctgggtagaaaatagatcgaaggtgggtaagggtagaatgaggaagcccagttcaggggccactgtggttatccaggcaagaggtgacagggcttcagccagtgtggaaacagctttgtgatttccgtgcagctgatcctgccaatgctaaatttcagatagcaactaggcattctacggattccaatcaccagtcttttcattattatatatcatatgttttatatattacacacacacacacacacacacacactctacacacacgtggttaagttgcttttgctagattttgttctataggagggatacctgatatattttaacaattaaataggttaggtttatgacaaaacaatttgacatgggttagacataatgtaatctttctatgctttaatacaatcttggcaaaaaacatttactcttgctgacaactcttctttggtttatttctctaagccacttagaatcctaaacatactaaatgacctgaaaaatgaggtgaggggggcataagaggcctactacccaagaaacaggtgtttggattgtttttccccccgtttagaaatttattacattgttccattagttatttttctctgctgacttgattggccaaagcaaacactgaaatccggcagaaagcactaagagactgctgatttatactttcaccaaaggtctcagacatcttagtgtccctgcatccttcctccatgtgatctccctaatgagcctcttgtcctgcttgcagccagacagtgggagccatgtgacagcttgcagctgaggctggacctcacccttgggggcagggctgcttctatgcacgaacagcatctcatttcatttattgctgtgatacataaatgccattttactctatcactggacttttttgttaaagtcatgctgttcaaaggaagcggtaaattagtctttttgttagtaaaatataaactgttttcctgttcctttatgatttttaaggaaattttaaactatagttgcattcctgagctcttcatttgttctgtcagaatggttcttgtcccactcagccacacagaatcaaagcaagtttcagggaagagcactcagtattccagatgaaggtagccatagtggaaggcctcagttttttcaccctctaagacggtgtctctcaaagtttagtgcgtggatctttggtgtgtggatcttttacattagaattccttgaggcttgttagaaatgcagattccttggtccctccccagaccaaatgactcattgtctccaagggtagggccaagaatctgcatggtagtcaatatccagggtattttcctgcatattcacatttgagaaccactactctaggttgtgtgactaggcaagaaaaatgcccctagaagattggccagctcacagcaagctctgcatggacttgttaaaaatggtgaatgttattcaaatgaaaactatgcttctaaggatttgttttcttcgagaaagtatgtcaccaccacattagtctcctcttccaactaaatcatctttcttctgtgcatttttgcctctgtttttggggattacagtggtcctatagcttaatcggctgatagttttgctgtgggtccaatttttgctgactttaggggggcactttgatcttgaagacggcttgaaatgatcattttgtctcatgtgccattttctcttttctttttgaacagcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

(19) United States
(12) Patent Application Publication
(10) Pub. No.: US 2020/0291407 A1
(43) Pub. Date: Sep. 17, 2020

(54) TREATMENT OF GLIAL CELL DERIVED NEUROTROPHIC FACTOR (GDNF) RELATED DISEASES BY INHIBITION OF NATURAL ANTISENSE TRANSCRIPT TO GDNF

(71) Applicant: CuRNA, Inc., Miami, FL (US)

(72) Inventors: Joseph COLLARD, Delray Beach, FL (US); Olga Khorkova Sherman, Tequesta, FL (US); Carlos Coito, West Palm Beach, FL (US)

(21) Appl. No.: 16/886,902

(22) Filed: May 29, 2020

Related U.S. Application Data
(63) Continuation of application No. 16/163,096, filed on Oct. 17, 2018.
Application No. 16/163,096 is a continuation of application No. 14/703,046, filed on May 4, 2015, now abandoned.
Application No. 14/703,046 is a continuation of application No. 13/759,278, filed on Feb. 5, 2013, now abandoned.
Application No. 13/759,278 is a continuation of application No. 13/201,260, filed on Sep. 13, 2011, now abandoned.
(60) Provisional application No. 61/152,239, filed on Feb. 12, 2009.

(51) Int. Cl. C12N 15/113; C12Q 1/6813

(52) U.S. Cl. CPC C12N 15/1136; C12N 2310/3533; C12Q 1/6813; C12N 2310/11; C12N 2310/113; C12N 2310/14; C12N 2310/311; C12N 2310/312; C12N 2310/313; C12N 2310/314; C12N 2310/315; C12N 2310/316; C12N 2310/3181; C12N 2310/321; C12N 2310/322; C12N 2310/3231; C12N 2310/3525; C12N 15/113

(57) ABSTRACT

The present invention relates to antisense oligonucleotides that modulate the expression of and/or function of Glial cell derived neurotrophic factor (GDNF), in particular, by targeting natural antisense polynucleotides of Glial cell derived neurotrophic factor (GDNF). The invention also relates to the identification of these antisense oligonucleotides and their use in treating diseases and disorders associated with the expression of GDNF.

The present application is a Continuation of U.S. application Ser. No. 16/163,096 filed Oct. 17, 2018, which is a Continuation of US application Ser. No. 14/703,046 filed May 4, 2015, which is a Continuation of U.S. application Ser. No. 13/759,278 filed Feb. 5, 2013, which is a Continuation of U.S. application Ser. No. 13/201,260 filed Sep. 13, 2011, which is a National Stage Entry of International Application No. PCT/US2010/024079 filed Feb. 12, 2010, which claims the benefit of U.S. Provisional Application No. 61/152,239 filed Feb. 12, 2009, which are all incorporated herein by reference in their entireties

### FIELD OF THE INVENTION

Embodiments of the invention comprise oligonucleotides modulating expression and/or function of GDNF and associated molecules.

### BACKGROUND

DNA-RNA and RNA-RNA hybridization are important to many aspects of nucleic acid function including DNA replication, transcription, and translation. Hybridization is also central to a variety of technologies that either detect a particular nucleic acid or alter its expression. Antisense nucleotides, for example, disrupt gene expression by hybridizing to target RNA, thereby interfering with RNA splicing, transcription, translation, and replication. Antisense DNA has the added feature that DNA-RNA hybrids serve as a substrate for digestion by ribonuclease H, an activity that is present in most cell types. Antisense molecules can be delivered into cells, as is the case for oligodeoxynucleotides (ODNs), or they can be expressed from endogenous genes as RNA molecules. The FDA recently approved an antisense drug, VITRAVENE™ (for treatment of cytomegalovirus retinitis), reflecting that antisense has therapeutic utility.

### SUMMARY

This Summary is provided to present a summary of the invention to briefly indicate the nature and substance of the invention. It is submitted with the understanding that it will not be used to interpret or limit the scope or meaning of the claims.

In one embodiment, the invention provides methods for inhibiting the action of a natural antisense transcript by using antisense oligonucleotide(s) targeted to any region of the natural antisense transcript resulting in up-regulation of the corresponding sense gene. It is also contemplated herein that inhibition of the natural antisense transcript can be achieved by siRNA, ribozymes and small molecules, which are considered to be within the scope of the present invention.

One embodiment provides a method of modulating function and/or expression of an GDNF polynucleotide in patient cells or tissues in vivo or in vitro comprising contacting said cells or tissues with an antisense oligonucleotide 5 to 30 nucleotides in length wherein said oligonucleotide has at least 50% sequence identity to a reverse complement of a polynucleotide comprising 5 to 30 consecutive nucleotides within nucleotides 1 to 237 of SEQ ID NO: 2 or nucleotides 1 to 1246 of SEQ ID NO: 3 or nucleotides 1 to 684 of SEQ ID NO: 4 (FIG. 3), or nucleotides 1 to 400 of SEQ ID NO: 42 or nucleotides 1 to 619 of SEQ ID NO: 43 or nucleotides 1 to 813 of SEQ ID NO: 44, thereby modulating function and/or expression of the GDNF polynucleotide in patient cells or tissues in vivo or In vitro.

In another preferred embodiment, an oligonucleotide targets a natural antisense sequence of GDNF polynucleotides, for example, nucleotides set forth in SEQ ID NOS: 2 to 4 and 42 to 44, and any variants, alleles, homologs, mutants, derivatives, fragments and complementary sequences thereto. Examples of antisense oligonucleotides are set forth as SEQ ID NOS: 5 to 34 (FIG. 4).

Another embodiment provides a method of modulating function and/or expression of an GDNF polynucleotide in patient cells or tissues in vive or in vitro comprising contacting said cells or tissues with an antisense oligonucleotide 5 to 30 nucleotides in length wherein said oligonucleotide has at least 50%/o sequence identity to a reverse complement of the an antisense of the GDNF polynucleotide; thereby modulating function and/or expression of the GDNF polynucleotide in patient cells or tissues in vivo or in vitro.

Another embodiment provides a method of modulating function and/or expression of at GDNF polynucleotide in patient cells or tissues in vivo or in vitro comprising contacting said cells or tissues with an antisense oligonucleotide 5 to 30 nucleotides in length wherein said oligonucleotide has at least 50% sequence identity to an antisense oligonucleotide to an GDNF antisense polynucleotide; thereby modulating function and/or expression of the GDNF polynucleotide in patient cells or tissues in vivo or in vitro.

In a preferred embodiment, a composition comprises one or more antisense oligonucleotides which bind to sense and/or antisense GDNF polynucleotides.

In another preferred embodiment, the oligonucleotides comprise one or more modified or substituted nucleotides.

In another preferred embodiment, the oligonucleotides comprise one or more modified bonds.

In yet another embodiment, the modified nucleotides comprise modified bases comprising phosphorothioate, methylphosphonate, peptide nucleic acids, 2-O-methyl, fluoro- or carbon, methylene or other locked nucleic acid (LNA) molecules. Preferably, the modified nucleotides are locked nucleic acid molecules, including α-L-LNA.

In another preferred embodiment, the oligonucleotides are administered to a patient subcutaneously, intramuscularly, intravenously or intraperitoneally.

In another preferred embodiment, the oligonucleotides are administered in a pharmaceutical composition. A treatment regimen comprises administering the antisense compounds at least once to patient; however, this treatment can be modified to include, multiple doses over a period of time. The treatment can be combined with one or more other types of therapies.

In another preferred embodiment, the oligonucleotides are encapsulated in a liposome or attached to a carrier molecule (e.g. cholesterol, TAT peptide).

Other aspects are described infra.

### BRIEF DESCRIPTION OF THE DRAWINGS

FIG. 1A: is a graph of real time PCR results showing the fold change+standard deviation in GDNF mRNA after treatment of HUVEC cells with phosphorothioate oligonucleotides introduced using Lipofectamine 2000, as compared to control. Bars denoted as CUR-0117, CUR-0118, CUR-0119. CUR-0120, CUR-0121 and CUR-0122 correspond to samples treated with SEQ ID NOS: 5 to 10 respectively.

FIG. 1B: is a graph of real time PCR results showing the fold change+standard deviation in GDNF mRNA after treatment of HepG2 cells with phosphorothioate oligonucleotides introduced using Lipofectamine 2000, as compared to control. Bars denoted as CUR-0741 to CUR-0764 correspond to samples treated with SEQ ID NOS: 11 to 34 respectively.

FIG. 1C: is a graph of real time PCR results showing the fold change+standard deviation in GDNF mRNA after treatment of Vero cells with phosphorothioate oligonucleotides introduced using Lipofectamine 2000, as compared to control. Bars denoted as CUR-0741 to CUR-0764 correspond to samples treated with SEQ ID NOS: 11 to 34 respectively.

FIG. 1D: is a graph of real time PCR results showing the fold change+standard deviation in GDNF mRNA after treatment of CHP212 cells with phosphorothioate oligonucleotides introduced using Lipofectamine 2000, as compared to control. Bars denoted as CUR-0751, CUR-0752, CUR-0753, CUR-0120, CUR-0121 and CUR-0117 correspond to samples treated with SEQ ID NOS: 21, 22, 23, 8, 9 and 5 respectively.

FIG. 2 shows SEQ ID NO: 1: Homo sapiens glial cell derived neurotrophic factor (GDNF), transcript variant 3. mRNA (NCBI accession number NM_199234.1) and SEQ ID NO: 45 shows the genomic sequence of GDNF (exons are shown in capital letters, introns in small).

FIG. 3 shows

SEQ ID NO: 2: Natural antisense sequence (AW883557.1 (A))

SEQ ID NO: 3: Natural antisense sequence (BM547433 (PR))

SEQ ID NO: 4: Natural antisense sequence (BX505687)

FIG. 4 shows the antisense oligonucleotides, SEQ ID NOs: 5 to 34. *indicates phosphothioate bond.

FIG. 5 shows SEQ ID NOS: 35 to 41.

FIG. 6 shows

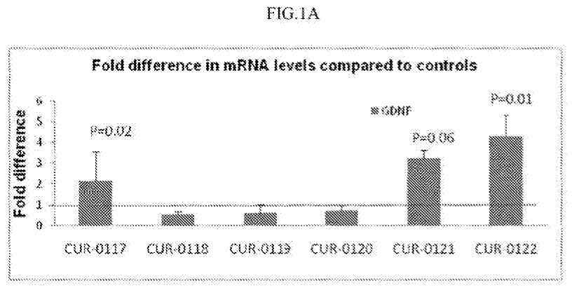

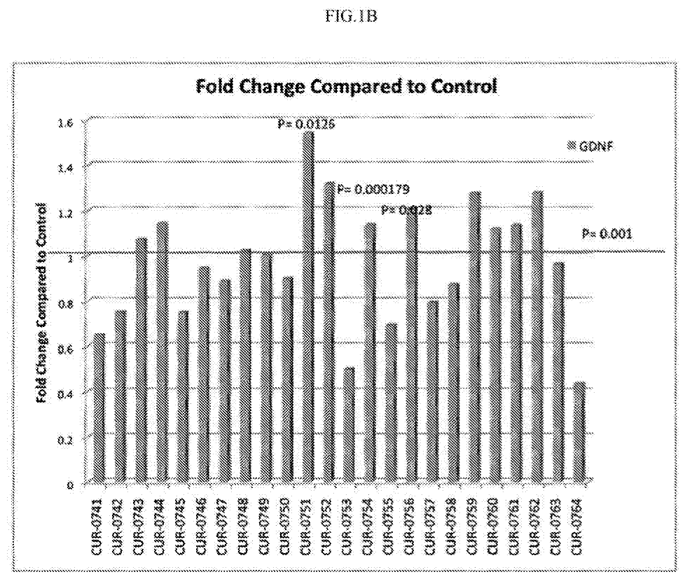

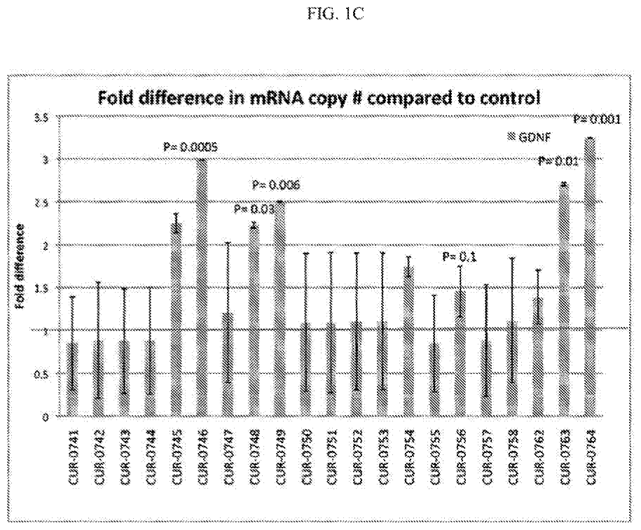

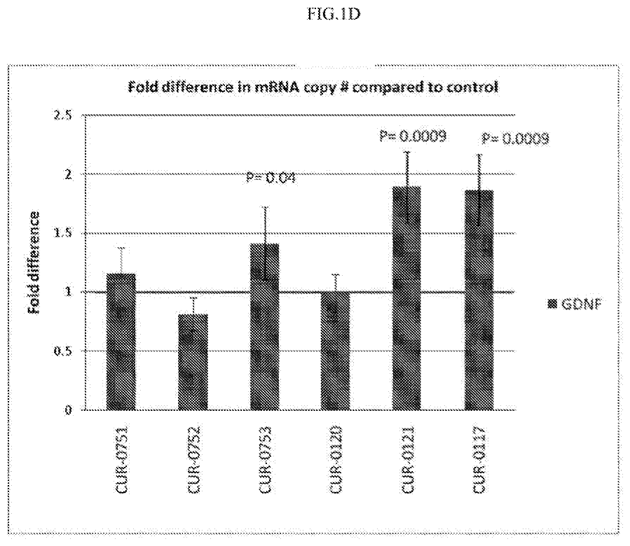

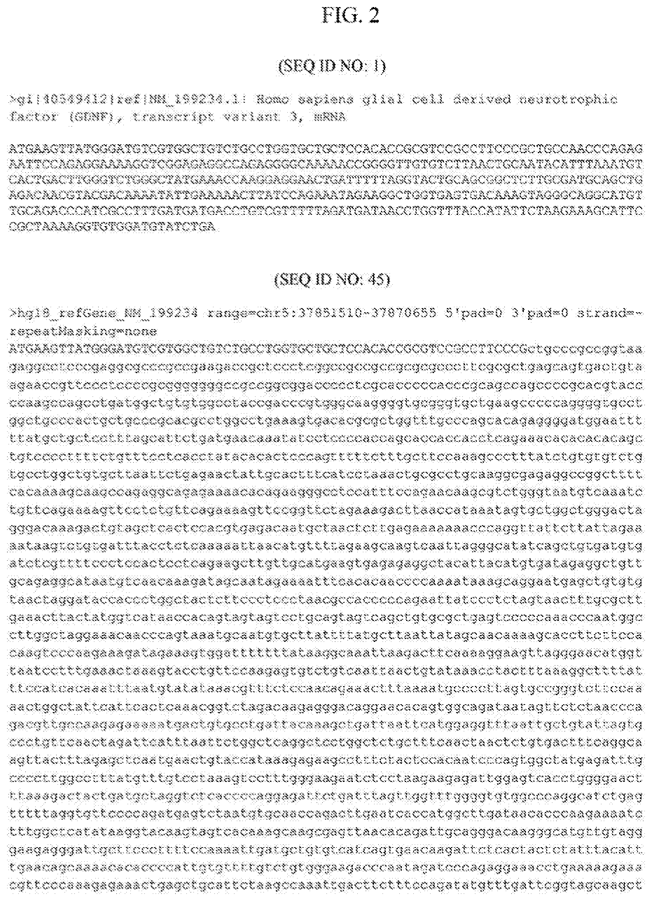

SEQ ID NO: 42: Natural antisense sequence (AW883557.1 (A)) alternate splicing a

SEQ ID NO: 43: Natural antisense sequence (AW883557.1 (A)) alternate splicing b

SEQ ID NO: 44: Natural antisense sequence (AW883557.1 (A)) alternate splicing c

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant an, however, will readily recognize that the invention can be practiced without one or more of the specific details or with other methods. The present invention is not limited by the ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively. “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “mRNA” means the presently known mRNA transcript(s) of a targeted gene, and any further transcripts which may be elucidated.

By “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA (Eguchi, et al., (1991) Ann. Rev. Biochem. 60, 631-652). An antisense oligonucleotide can upregulate or downregulate expression and/or function of a particular polynucleotide. The definition is meant to include any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

In the context of this invention, the term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoögsteen types of base pairing, or the like.

The oligonucleotide may be “chimeric”, that is, composed of different regions. In the context of this invention “chimeric” compounds are oligonucleotides, which contain two or more chemical regions, for example, DNA region(s) RNA region(s), PNA region(s) etc. Each chemical region is made up of at least one monomer unit, i.e., a nucleotide in the case of an oligonucleotides compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or to oligonucleotide analogs as described above.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein “GDNF” and “Glial cell derived neurotrophic factor” are inclusive of all family members, mutants, alleles, fragments, species, coding and noncoding sequences, sense and antisense polynucleotide strands, etc.

As used herein, the words ‘Glial cell derived neurotrophic factor’, ‘Glial cell line-derived neurotrophic factor’, ‘Glial cell-derived neurotrophic factor’, ‘Astrocyte-derived trophic factor’, ‘ATF’, ‘ATF1’, ‘ATF2’, ‘HFB1-GDNF’, ‘hGDNF’ and GDNF are considered the same in the literature and are used interchangeably in the present application.

As used herein, the term “oligonucleotide specific for” or “oligonucleotide which targets” refers to an oligonucleotide having a sequence (i) capable of forming a stable complex with a portion of the targeted gene, or (ii) capable of forming a stable duplex with a portion of a mRNA transcript of the targeted gene. Stability of the complexes and duplexes can be determined by theoretical calculations and/or in vitro assays. Exemplary assays for determining stability of hybridization complexes and duplexes are described in the Examples below.

As used herein, the term “target nucleic acid” encompasses DNA, RNA (comprising premRNA and mRNA) transcribed from such DNA, and also cDNA derived from such RNA, coding, noncoding sequences, sense or antisense polynucleotides. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered include, for example, replication and transcription. The functions of RNA to be interfered, include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of an encoded product or oligonucleotides.

RNA interference “RNAi” is mediated by double stranded RNA (dsRNA) molecules that have sequence-specific homology to their “target” nucleic acid sequences (Caplen, N. J., et al. (2001) Proc. Natl. Acad. Sc. USA 98:9742-9747). In certain embodiments of the present invention, the mediators are 5-25 nucleotide “small interfering” RNA duplexes (siRNAs). The siRNAs are derived from the processing of dsRNA by an RNase enzyme known as Dicer (Bernstein, E., et al. (2001) Nature 409:363-366). siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al. (2001) Nature 409:363-366; Boutla, A., et al. (2001) Curr. Biol. 11:1776-1780). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 1 to about 50 nucleotides (nt). In examples of non limiting embodiments, siRNAs can comprise about 5 to about 40 nt, about 5 to about 30 nt, about 10 to about 30 nt, about 15 to about 25 nt, or about 20-25 nucleotides.

Selection of appropriate oligonucleotides is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

By “enzymatic RNA” is meant an RNA molecule with enzymatic activity (Cech, (1988) J. American. Med. Assoc. 260, 3030-3035). Enzymatic nucleic acids (ribozymes) act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through base pairing, and once bound to the correct site, acts enzymatically to cut the target RNA.

By “decoy RNA” is meant an RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al. (990) Cell, 63, 601-608). This is meant to be a specific example. Those in the art will recognize that this is but one example, and other embodiments can be readily generated using techniques generally known in the art.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphomates, phosphoroselenoate, phosphoramidate, and the like, as more fully described below.

The term “nucleotide” covers naturally occurring nucleotides as well as nonnaturally occurring nucleotides. It should be clear to the person skilled in the art that various nucleotides which previously have been considered “non-naturally occuring” have subsequently been found in nature. Thus, “nucleotides” includes not only the known purine and pyrimidine heterocycles-containing molecules, but also heterocyclic analogues and tautomers thereof. Illustrative examples of other types of nucleotides are molecules containing adenine, guanine, thymine, cytosine, uracil, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleotides described in Benner et al., U.S. Pat. No. 5,432,272. The term “nucleotide” is intended to cover every and all of these examples as well as analogues and tautomers thereof. Especially interesting nucleotides are those containing adenine, guanine, thymine, cytosine, and uracil, which are considered as the naturally occurring nucleotides in relation to therapeutic and diagnostic application in humans. Nucleotides include the natural 2′-deoxy and 2′-hydroxyl sugars, e.g., as described in Kornberg and Baker, DNA Replication, 2nd Ed. (Freeman, San Francisco, 1992) as well as their analogs.

“Analogs” in reference to nucleotides includes synthetic nucleotides having modified base moieties and/or modified sugar moieties (see e.g., described generally by Scheit. Nucleotide Analogs, John Wiley, New York, 1980; Freier & Altmann, (1997) Nucl. Acid. Res., 25(22), 4429-4443, Toulmé, J. J., (2001) Nature Biotechnology 19:17-18, Manoharan M., (1999) Biochemica Biophysica Acta 1489:117-139; Freier S. M., (1997) Nucleic Acid Research, 25:4429-4443, Uhlman, E., (2000) Drug Discovery & Development, 3: 203-213, Herdewin P., (2000) Antisense & Nucleic Acid Drug Dev., 10:297-310); 2′-O, 3′-C-linked [3.2.0] bicycloarabinonucleosides (see N. K Christiensen, et al, (1998) J. Am. Chem. Soc., 120: 5458-5463; Prakash T P, Bhat B. (2007) Curr Top Med Chem. 7(7):641-9; Cho E J, et al. (2009) Annual Review of Analytical Chemistry, 2, 241-264). Such analogs include synthetic nucleotides designed to enhance binding properties, e.g., duplex or triplex stability, specificity, or the like.

As used herein, “hybridization” means the pairing of substantially complementary strands of oligomeric compounds. One mechanism of pairing involves hydrogen bonding, which may be Watson-Crick, Hoögsteen or reversed Hoögsteen hydrogen bonding, between complementary nucleoside or nucleotide bases (nucleotides) of the strands of oligomeric compounds. For example, adenine and thymine are complementary nucleotides which pair through the formation of hydrogen bonds. Hybridization can occur under varying circumstances.

An antisense compound is “specifically hybridizable” when binding of the compound to the target nucleic acid interferes with the normal function of the target nucleic acid to cause a modulation of function and/or activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated. In general, stringent hybridization conditions comprise low concentrations (<0.15M) of salts with inorganic cations such as Na++ or K++ (i.e., low ionic strength), temperature higher than 20° C.-25° C. below the Tm of the oligomeric compound:target sequence complex, and the presence of denaturants such as formamide, dimethylformamide, dimethyl sulfoxide, or the detergent sodium dodecyl sulfate (SDS). For example, the hybridization rate decreases 1.1% for each 1% formamide. An example of a high stringency hybridization condition is 0.1× sodium chloride-sodium citrate buffer (SSC)/0.1% (w/v) SDS at 60° C., for 30 minutes.

“Complementary,” as used herein, refers to the capacity for precise pairing between two nucleotides on one or two oligomeric strands. For example, if a nucleobase at a certain position of an antisense compound is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligomeric compound and the further DNA, RNA, or oligonucleotide molecule are complementary to each other when a sufficient number of complementary positions in each molecule are occupied by nucleotides which can hydrogen bond with each other. Thus, “specifically hybridizable” and “complementary” are terms which are used to indicate a sufficient degree of precise pairing or complementarity over a sufficient number of nucleotides such that stable and specific binding occurs between the oligomeric compound and a target nucleic acid.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The oligomeric compounds of the present invention comprise at least about 70%, or at least about 75%, or at least about 80%, or at least about 85%, or at least about 90%, or at least about 95%, or at least about 99% sequence complementarity to a target region within the target nucleic acid sequence to which they are targeted. For example, an antisense compound in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, an antisense compound which is 18 nucleotides in length having 4 (four) noncomplementary nucleotides which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., (1990) J. Mol. Biol., 215, 403-410; Zhang and Madden, (1997) Genome Res., 7, 649-656). Percent homology, sequence identity or complementarity, can be determined by, for example, the Gap program (Wisconsin Sequence Analysis Package, Version 8 for Unix. Genetics Computer Group, University Research Park, Madison Wis.), using default settings, which uses the algorithm of Smith and Waterman (Adv. Appl. Math., (1981) 2, 482-489).

As used herein, the term “Thermal Melting Point (Tm)” refers to the temperature, under defined ionic strength, pH, and nucleic acid concentration, at which 50% of the oligonucleotides complementary to the target sequence hybridize to the target sequence at equilibrium. Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

As used herein, “modulation” means either an increase (stimulation) or a decrease (inhibition) in the expression of a gene.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide poly-morphisms” (SNPs,) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

As used herein, the term “animal” or “patient” is meant to include, for example, humans, sheep, elks, deer, mule deer, minks, mammals, monkeys, horses, cattle, pigs, goats, dogs, cats, rats, mice, birds, chicken, reptiles, fish, insects and arachnids.

“Mammal” covers warm blooded mammals that are typically under medical care (e.g., humans and domesticated animals). Examples include feline, canine, equine, bovine, and human, as well as just human.

“Treating” or “treatment” covers the treatment of a disease-state in a mammal, and includes: (a) preventing the disease-state from occurring in a mammal, in particular, when such mammal is predisposed to the disease-state but, has not yet been diagnosed as having it (b) inhibiting the disease-state, e.g., arresting it development; and/or (c) relieving the disease-state. e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

As used herein, the term “cancer” refers to any malignant tumor, particularly arising in the lung, kidney, or thyroid. The cancer manifests itself as a “tumor” or tissue comprising malignant cells of the cancer. Examples of tumors include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. As noted above, the invention specifically permits differential diagnosis of lung, kidney, and thyroid tumors.

### Polynucleotide and Oligonucleotide Compositions and Molecules

Targets:

In one embodiment, the targets comprise nucleic acid sequences of Glial cell derived neurotrophic factor (GDNF), including without limitation sense and/or antisense noncoding and/or coding sequences associated with GDNF.

The glial derived GDNF family of nerotrophic factors includes four members: glial cell line-derived neurotrophic factor (GDNF), neurturin, artemin and persephin (PSPN). GDNF family ligands signal through receptors consisting of a GPI-linked GFRα subunit and the transmembrane receptor tyrosine kinase RET. In order to activate the transmembrane receptor tyrosine kinase Ret, each of the GDNF family neurotrophic factors binds preferentially to one of the glycosyl-phosphatidylinositol (GPI)-linked GDNF family α-receptors (GFRα1-4). GDNF is a protein that may be identified in or obtained from glial cells and that exhibits neurotrophic activity. More specifically, GDNF is a dopiminergic neurotrophic protein that is characterized in part by its ability to increase dopamine uptake on the embryonic precursors of the substantia nigra dopinergic neurons, and further by its ability to promote the survival of parasympathetic and sympathetic nerve cells.

In preferred embodiments, antisense oligonucleotides are used to prevent or treat diseases or disorders associated treatment of diseases associated to an increase or reduction of the activity of decoupling proteins. Examples of diseases which can be treated with cell/tissues regenerated from stem cells obtained using the antisense compounds comprise a disease or a disorder associated with defective neurogenesis; a neurodegenerative disease or disorder (e.g., Alzheimer's disease, Parkinson's disease, Huntington's disease, amyotrophic lateral sclerosis etc.): a neuropsychiatric disorder (depression, schizophrenia, schizofreniform disorder, schizoaffective disorder, and delusional disorder; anxiety disorders such as panic disorder, phobias (including agoraphobia), an obsessive-compulsive disorder, a posttraumatic stress disorder, a bipolar disorder, anorexia nervosa, bulimia nervosa, an autoimmune disorder (e.g., multiple sclerosis) of the central nervous system, memory loss, a long term or a short term memory disorder, benign forgetfulness, a childhood learning disorder, close head injury, an attention deficit disorder, neuronal reaction to viral infection, brain damage, narcolepsy, a sleep disorder (e.g., circadian rhythm disorders, insomnia and narcolepsy); severance of nerves or nerve damage, severance of cerebrospinal nerve cord (CNS) and a damage to brain or nerve cells, a neurological deficit associated with AIDS, a motor and tic disorder characterized by motor and/or vocal tics (e.g., Tourette's disorder, chronic motor or vocal tic disorder, transient tic disorder, and stereotypic movement disorder), a substance abuse disorder (e.g., substance dependence, substance abuse and the sequalae of substance abuse/dependence, such as substance-induced psychological disorder, substance withdrawal and substance-induced dementia or amnestic disorder), traumatic brain injury, tinnitus, neuralgia (e.g., trigeminal neuralgia) pain (e.g. chronic pain, chronic inflammatory pain, pain associated with arthritis, fibromyalgia, back pain, cancer-associated pain, pain associated with digestive disease, pain associated with Crohn's disease, pain associated with autoimmune disease, pain associated with endocrine disease, pain associated with diabetic neuropathy, phantom limb pain, spontaneous pain, chronic post-surgical pain, chronic, temporomandibular pain, causalgia, post-herpetic neuralgia, AIDS-related pain, complex regional pain syndromes type I and II, trigeminal neuralgia, chronic back pain, pain associated with spinal cord injury, pain associated with drug intake and recurrent acute pain, neuropathic pain), inappropriate neuronal activity resulting in neurodysthesias in a disease such as diabetes, an MS and a motor neuron disease, ataxias muscular rigidity (spasticity), temporomandibular joint dysfunction. Reward deficiency syndrome (RDS), neurotoxicity caused by alcohol or substance abuse (e.g., ecstacy, methamphetamine etc.), mental retardation or cognitive impairment (e.g., nonsyndromic X-linked mental retardation, fragile X syndrome, Down's syndrome, autism), aphasia, Bell's palsy, Creutzfeldt-jacob disease, encephalitis, age related macular degeneration, ondine syndrome, WAGR syndrome, hearing loss, Werdnig-Hoffmann disease, chronic proximal spinal muscular atrophy, Guillain-Barre syndrome, Multiple System Atrophy (Shy Drager Syndrome), Rett syndrome, epilepsy, spinal cord injury, stroke, hypoxia, ischemia, brain injury, diabetic neuropathy, a kidney disease or renal dysfunction, peripheral neuropathy, nerve transplantation complications, motor neuron disease, peripheral nerve injury, obesity, a metabolic syndrome, cancer, eczema, a disorder of intestinal motility, Hirschsprung's disease, Achalasia, Esophageal spasm, Scleroderma (related to muscular atrophy of the smooth muscle portion of the esophagus, weakness of contraction of the lower two-thirds of the esophageal body, and incompetence of the lower esophageal sphincter, but also caused by treatment with immunosuppressive agents), duodenal ulcer, Zollinger-Ellison Syndrome, hypersecretion of gastric acid, malabsorptive disorder, an epidermal and stromal wound healing disorder and/or a scarring disorder, a progressive muscular dystrophy (e.g., Duchenne, Becker, Emery-Dreifuss, Landouzy-Dejerine, scapulohumeral, limb-girdle, Von Graefe-Fuchs, oculopharyngeal, myotonic and congenital), a congenital or acquired myopathy, anemia (including macrocytic and aplastic anemia); thrombocytopenia; hypoplasia; disseminated intravascular coagulation (DIC); myelodysplasia; immune (autoimmune) thrombocytopenic purpura (ITP), HIV induced ITP, a thrombocytotic disease, a viral infection, a neuro-oncological disease or disorder, neuro-immunological disease or disorder and neuro-otological disease or disorder, cochlear sensory cell damage, defective auditory perception, phaeochromocytoma, multiple endocrine neoplasia type 2, von Hippel-Lindau disease (VHL), type 1 neurofibromatosis; and a disease or disorder associated with aging and senescence.

In a preferred embodiment, the oligonucleotides are specific for polynucleotides of GDNF, which includes, without limitation noncoding regions. The GDNF targets comprise variants of GDNF; mutants of GDNF, including SNPs; noncoding sequences of GDNF; alleles, fragments and the like. Preferably the oligonucleotide is an antisense RNA molecule.

In accordance with embodiments of the invention, the target nucleic acid molecule is not limited to GDNF polynucleotides alone but extends to any of the isoforms, receptors, homologs, non-coding regions and the like of GDNF.

In another preferred embodiment, an oligonucleotide targets a natural antisense sequence (natural antisense to the coding and non-coding regions) of GDNF targets, including, without limitation, variants, alleles, homologs, mutants, derivatives, fragments and complementary sequences thereto. Preferably the oligonucleotide is an antisense RNA or DNA molecule.

In another preferred embodiment, the oligomeric compounds of the present invention also include variants in which a different base is present at one or more of the nucleotide positions in the compound. For example, if the first nucleotide is an adenine, variants may be produced which contain thymidine, guanosine, cytidine or other natural or unnatural nucleotides at this position. This may be done at any of the positions of the antisense compound. These compounds are then tested using the methods described herein to determine their ability to inhibit expression of a target nucleic acid.

In some embodiments, homology, sequence identity or complementarity, between the antisense compound and target is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired. Such conditions include, i.e., physiological conditions in the case of in vivo assays or therapeutic treatment, and conditions in which assays are performed in the case of in vitro assays.

An antisense compound, whether DNA, RNA, chimeric, substituted etc, is specifically hybridizable when binding of the compound to the target DNA or RNA molecule interferes with the normal function of the target DNA or RNA to cause a loss of utility, and there is a sufficient degree of complementarily to avoid non-specific binding of the antisense compound to non-target sequences under conditions in which specific binding is desired, i.e., under physiological conditions in the case of in vivo assays or therapeutic treatment, and in the case of n vitro assays, under conditions in which the assays are performed.

In another preferred embodiment, targeting of GDNF including without limitation, antisense sequences which are identified and expanded, using for example, PCR, hybridization etc., one or more of the sequences set forth as SEQ ID NOS: 2, 3 or 4, and the like, modulate the expression or function of GDNF. In one embodiment, expression or function is up-regulated as compared to a control. In another preferred embodiment, expression or function is down-regulated as compared to a control.

In another preferred embodiment, oligonucleotides comprise nucleic acid sequences set forth as SEQ ID NOS: 5 to 34 including antisense sequences which are identified and expanded, using for example, PCR, hybridization etc. These oligonucleotides can comprise one or more modified nucleotides, shorter or longer fragments, modified bonds and the like. Examples of modified bonds or internucleotide linkages comprise phosphorothioate, phosphorodithioate or the like. In another preferred embodiment, the nucleotides comprise a phosphorus derivative. The phosphorus derivative (or modified phosphate group) which may be attached to the sugar or sugar analog moiety in the modified oligonucleotides of the present invention may be a monophosphate, diphosphate, triphosphate, alkylphosphate, alkanephosphate, phosphorothioate and the like. The preparation of the above-noted phosphate analogs, and their incorporation into nucleotides, modified nucleotides and oligonucleotides, per se, is also known and need not be described here.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In embodiments of the present invention oligomeric antisense compounds, particularly oligonucleotides, bind to target nucleic acid molecules and modulate the expression and/or function of molecules encoded by a target gene. The functions of DNA to be interfered comprise, for example, replication and transcription. The functions of RNA to be interfered comprise all vital functions such as, for example, translocation of the RNA to the site of protein translation, translation of protein from the RNA, splicing of the RNA to yield one or more mRNA species, and catalytic activity which may be engaged in or facilitated by the RNA. The functions may be up-regulated or inhibited depending on the functions desired.

The antisense compounds, include, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

Targeting an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the present invention, the target nucleic acid encodes Glial cell derived neurotrophic factor (GDNF).

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

In a preferred embodiment, the antisense oligonucleotides hind to the natural antisense sequences of Glial cell derived neurotrophic factor (GDNF) and modulate the expression and/or function of Glial cell derived neurotrophic factor (GDNF) (SEQ ID NO: i). Examples of antisense sequences include SEQ ID NOS: 2 to 34.

Table 1 shows exemplary antisense oligonucleotides useful in the methods of the present invention.

TABLE 1

| | Oli- | |
|---|---|---|
| Seq | go | |
| ID | Name | Sequence |
| SEQ | CUR- | C*A*C*C*C*T*G*G*C*T*A*C*T*C*T*T*C*C*C*T |
| ID | 0117 | |
| NO: | | |
| 5 | | |
| SEQ: | CUR- | G*G*C*T*A*C*T*C*T*T*C*C*C*T*C*C*C*T*A |
| ID | 0118 | |
| NO: | | |
| 6 | | |
| SEQ | CUR- | T*G*T*G*T*G*T*G*T*G*T*G*T*G*T*G*T*G*T*G*T |
| ID | 0119 | |
| NO: | | |
| 7 | | |
| SEQ | CUR- | T*T*C*T*A*C*C*C*T*T*A*C*C*C*A*C*C*T*T*C |
| ID | 0120 | |
| NO: | | |
| 8 | | |
| SEQ | CUR- | G*T*C*G*C*C*T*T*G*C*C*T*T*C*C*C*A*T*A*C |
| ID | 0121 | |
| NO: | | |
| 9 | | |
| SEQ | CUR- | G*G*T*G*G*G*T*N*T*G*G*A*A*G*T*G*G*G*A*T |
| ID | 0122 | |
| NO: | | |
| 10 | | |
| SEQ | CUR- | c*g*g*c*a*g*c*c*c*t*c*g*c |
| ID | 0741 | |
| NO: | | |
| 11 | | |
| SEQ | CUR- | t*g*g*g*g*g*t*g*c*g*g*g*g*g |
| ID | 0742 | |
| NO: | | |
| 12 | | |
| SEQ | CUR- | g*g*a*c*c*t*c*g*g*c*t*t*c*t |
| ID | 0743 | |
| NO: | | |
| 13 | | |
| SEQ | CUR- | g*c*g*g*c*g*g*c*t*g*c*t*c*g |
| ID | 0744 | |
| NO: | | |
| 14 | | |
| SEQ | CUR- | c*c*a*c*c*c*a*a*a*g*c*a*g*c |
| ID | 0745 | |
| NO: | | |
| 15 | | |
| SEQ | CUR- | c*c*c*c*c*c*a*c*c*c*a*a*a*g |
| ID | 0746 | |
| NO: | | |
| 16 | | |
| SEQ | CUR- | g*c*g*c*a*g*c*c*c*t*g*t*c*a |
| ID | 0747 | |
| NO: | | |
| 17 | | |
| SEQ | CUR- | c*g*c*g*c*g*c*a*g*c*c*c*t*g |
| ID | 0748 | |
| NO: | | |
| 18 | | |
| SEQ | CUR- | c*a*g*c*c*a*a*g*a*g*c*g*c*g |
| ID | 0749 | |
| NO: | | |
| 19 | | |
| SEQ | CUR- | g*g*c*c*c*g*c*g*c*a*g*c*c*c |
| ID | 0750 | |
| NO: | | |
| 20 | | |
| SEQ | CUR- | g*c*c*c*g*c*a*g*c*g*c*c*c*c*g |
| ID | 0751 | |
| NO: | | |
| 21 | | |
| SEQ | CUR- | g*a*g*g*c*g*c*a*g*a*g*c*g*c |
| ID | 0752 | |
| NO: | | |
| 22 | | |
| SEQ | CUR- | c*a*g*t*g*c*g*c*c*c*a*g*a*g |
| ID | 0753 | |
| NO: | | |
| 23 | | |
| SEQ | CUR- | g*t*g*c*t*c*c*c*a*g*g*c*a*g |
| ID | 0754 | |
| NO: | | |
| 24 | | |
| SEQ | CUR- | c*t*g*c*c*t*g*g*g*a*g*c*a*c |
| ID | 0755 | |
| NO: | | |
| 25 | | |
| SEQ | CUR- | a*a*g*a*c*c*t*c*a*g*c*t*c*c |
| ID | 0756 | |
| NO: | | |
| 26 | | |
| SEQ | CUR- | t*t*c*g*g*a*t*c*t*c*c*a*g*g*c |
| ID | 0757 | |
| NO: | | |
| 27 | | |
| SEQ | CUR- | t*g*a*c*g*t*g*g*t*g*t*c*t*c |
| ID | 0758 | |
| NO: | | |
| 28 | | |
| SEQ | CUR- | c*t*c*c*c*c*g*c*g*c*c*g*g*t |
| ID | 0759 | |
| NO: | | |
| 29 | | |
| SEQ | CUR- | a*t*g*t*c*t*t*c*a*c*g*g*g*a |
| ID | 0760 | |
| NO: | | |
| 30 | | |
| SEQ | CUR- | c*t*c*c*t*g*g*c*g*c*c*c*t*c |
| ID | 0761 | |
| NO: | | |
| 31 | | |
| SEQ | CUR- | a*a*g*a*c*c*a*g*c*c*t*g*c*g |
| ID | 0762 | |
| NO: | | |
| 32 | | |
| SEQ | CUR- | g*c*t*c*t*a*g*a*a*g*a*c*c*a |
| ID | 0763 | |
| NO: | | |
| 33 | | |
| SEQ | CUR- | c*c*t*c*c*c*c*c*a*c*g*c |
| ID | 0764 | |
| NO: | | |
| 34 | | |

In another preferred embodiment, the antisense oligonucleotides bind to one or more segments of Glial cell derived neurotrophic factor (GDNF) polynucleotides and modulate the expression and/or function of Glial cell derived neurotrophic factor (GDNF). The segments comprise at least five consecutive nucleotides of the Glial cell derived neurotrophic factor (GDNF) sense or antisense polynucleotides.

In another preferred embodiment, the antisense oligonucleotides are specific for natural antisense sequences of Glial cell derived neurotrophic factor (GDNF) wherein binding of the oligonucleotides to the natural antisense sequences of Glial cell derived neurotrophic factor (GDNF) modulate expression and/or function of Glial cell derived neurotrophic factor (GDNF).

In another preferred embodiment, oligonucleotide compounds comprise sequences set forth as SEQ ID NOS: 5 to 34, antisense sequences which are identified and expanded, using for example, PCR, hybridization etc These oligonucleotides can comprise one or more modified nucleotides, shorter or longer fragments, modified bonds and the like. Examples of modified bonds or internucleotide linkages comprise phosphorothioate, phosphorodithioate or the like. In another preferred embodiment, the nucleotides comprise a phosphorus derivative. The phosphorus derivative (or modified phosphate group) which may be attached to the sugar or sugar analog moiety in the modified oligonucleotides of the present invention may be a monophosphate, diphosphate, triphosphate, alkylphosphate, alkanephosphate, phosphorothioate and the like. The preparation of the above-noted phosphate analogs, and their incorporation into nucleotides, modified nucleotides and oligonucleotides, per se, is also known and need not be described here.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes has a translation initiation codon having the RNA sequence 5′-GUG, 5′-UUG or 5′-CUG; and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in to initiate translation of an mRNA transcribed from a gene encoding Glial cell derived neurotrophic factor (GDNF), regardless of the sequence(s) of such codons. A translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions that may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a targeted region is the intragenic region encompassing the translation initiation or termination codon of the open reading frame (ORF) of a gene.

Another target region includes the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene). Still another target region includes the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. Another target region for this invention is the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. In one embodiment, targeting splice sites, i.e., intron-exon junctions or exon-intron junctions, is particularly useful in situations where aberrant splicing is implicated in disease, or where an overproduction of a particular splice product is implicated in disease. An aberrant fusion junction due to rearrangement or deletion is another embodiment of a target site. mRNA transcripts produced via the process of splicing of two (or more) mRNAs from different gene sources are known as “fusion transcripts”. Introns can be effectively targeted using antisense compounds targeted to, for example, DNA or pre-mRNA

In another preferred embodiment, the antisense oligonucleotides bind to coding and/or to non-coding regions of a target polynucleotide and modulate the expression and/or function of the target molecule.

In another preferred embodiment, the antisense oligonucleotides bind to natural antisense polynucleotides and modulate the expression and/or function of the target molecule.

In another preferred embodiment, the antisense oligonucleotides bind to sense polynucleotides and modulate the expression and/or function of the target molecule.

Alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

Variants can be produced through the use of alternative signals to start or stop transcription. Pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also embodiments of target nucleic acids.

The locations on the target nucleic acid to which the antisense compounds hybridize are defined as at least a 5-nucleotide long portion of a target region to which an active antisense compound is targeted.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure.

Target segments 5-100 nucleotides in length comprising a stretch of at least five (5) consecutive nucleotides selected from within the illustrative preferred target segments are considered to be suitable for targeting as well.

Target segments can include DNA or RNA sequences that comprise at least the 5 consecutive nucleotides from the 5′-terminus of one of the illustrative preferred target segments (the remaining nucleotides being a consecutive stretch of the same DNA or RNA beginning immediately upstream of the 5′-terminus of the target segment and continuing until the DNA or RNA contains about 5 to about 100 nucleotides). Similarly preferred target segments are represented by DNA or RNA sequences that comprise at least the 5 consecutive nucleotides from the 3′-terminus of one of the illustrative preferred target segments (the remaining nucleotides being a consecutive stretch of the same DNA or RNA beginning immediately downstream of the 3′-terminus of the target segment and continuing until the DNA or RNA contains about 5 to about 100 nucleotides). One having skill in the art armed with the target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

In embodiments of the invention the oligonucleotides bind to an antisense strand of a particular target. The oligonucleotides are at least 5 nucleotides in length and can be synthesized so each oligonucleotide targets overlapping sequences such that oligonucleotides are synthesized to cover the entire length of the target polynucleotide. The targets also include coding as well as non coding regions.

In one embodiment, it is preferred to target specific nucleic acids by antisense oligonucleotides. Targeting an antisense compound to a particular nucleic acid, is a multistep process. The process usually begins with the identification of a nucleic acid sequence whose function is to be modulated. This may be, for example, a cellular gene (or mRNA transcribed from the gene) whose expression is associated with a particular disorder or disease state, or a non coding polynucleotide such as for example, non coding RNA (ncRNA).

RNAs can be classified into (1) messenger RNAs (mRNAs), which are translated into proteins, and (2) non-protein-coding RNAs (ncRNAs), ncRNAs comprise microRNAs, antisense transcripts and other Transcriptional Units (TU) containing a high density of stop codons and lacking any extensive “Open Reading Frame”. Many ncRNAs appear to start from initiation sites in 3′ untranslated regions (3′UTRs) of protein-coding loci. ncRNAs are often rare and at least half of the ncRNAs that have been sequenced by the FANTOM consortium seem not to be polyadenylated. Most researchers have for obvious reasons focused on polyadenylated mRNAs that are processed and exported to the cytoplasm. Recently, it was shown that the set of non-polyadenylated nuclear RNAs may be very large, and that many such transcripts arise from so-called intergenic regions (Cheng. J. et al. (2005) Science 308 (5725), 1149-1154; Kapranov, P. et al. (2005). Genome Res 15 (7), 987-997). The mechanism by which ncRNAs may regulate gene expression is by base pairing with target transcripts. The RNAs that function by base pairing can be grouped into (1) cis encoded RNAs that are encoded at the same genetic location, but on the opposite strand to the RNAs they act upon and therefore display perfect complementarity to their target, and (2) trans-encoded RNAs that are encoded at a chromosomal location distinct from the RNAs they act upon and generally do not exhibit perfect base-pairing potential with their targets.

Without wishing to be bound by theory, perturbation of an antisense polynucleotide by the antisense oligonucleotides described herein can alter the expression of the corresponding sense messenger RNAs. However, this regulation can either be discordant (antisense knockdown results in messenger RNA elevation) or concordant (antisense knockdown results in concomitant messenger RNA reduction). In these cases, antisense oligonucleotides can be targeted to overlapping or non-overlapping parts of the antisense transcript resulting in its knockdown or sequestration. Coding as well as non-coding antisense can be targeted in an identical manner and that either category is capable of regulating the corresponding sense transcripts—either in a concordant or disconcordant manner. The strategies that are employed in identifying new oligonucleotides for use against a target can be based on the knockdown of antisense RNA transcripts by antisense oligonucleotides or any other means of modulating the desired target.

Strategy 1:

In the case of discordant regulation, knocking down the antisense transcript elevates the expression of the conventional (sense) gene. Should that latter gene encode for a known or putative drug target, then knockdown of its antisense counterpart could conceivably mimic the action of a receptor agonist or an enzyme stimulant.

Strategy 2:

In the case of concordant regulation, one could concomitantly knock down both antisense and sense transcripts and thereby achieve synergistic reduction of the conventional (sense) gene expression. If, for example, an antisense oligonucleotide is used to achieve knockdown, then this strategy can be used to apply one antisense oligonucleotide targeted to the sense transcript and another antisense oligonucleotide to the corresponding antisense transcript, or a single energetically symmetric antisense oligonucleotide that simultaneously targets overlapping sense and antisense transcripts.

According to the present invention, antisense compounds include antisense oligonucleotides, ribozymes, external guide sequence (EGS) oligonucleotides, siRNA compounds, single- or double-stranded RNA interference (RNAi) compounds such as siRNA compounds, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, doublestranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleotide positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines, however, in some embodiments, the gene expression or function is up regulated. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

Once introduced to a system, the compounds of the invention may elicit the action of one or more enzymes or structural proteins to effect cleavage or other modification of the target nucleic acid or may work via occupancy-based mechanisms, in general, nucleic acids (including oligonucleotides) may be described as “DNA-like” (i.e., generally having one or more 2′-deoxy sugars and, generally, T rather than U bases) or “RNA-like” (i.e., generally having one or more 2′-hydroxyl or 2′-modified sugars and, generally U rather than T bases). Nucleic acid helices can adopt more than one type of structure, most commonly the A- and B-forms. It is believed that, in general, oligonucleotides which have B-form-like structure are “DNA-like” and those which have A-formlike structure are “RNA-like.” In some (chimeric) embodiments, an antisense compound may contain both A- and B-form regions.

In another preferred embodiment, the desired oligonucleotides or antisense compounds, comprise at least one of: antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

dsRNA can also activate gene expression, a mechanism that has been termed “small RNA-induced gene activation” or RNA. dsRNAs targeting gene promoters induce potent transcriptional activation of associated genes. RNAa was demonstrated in human cells using synthetic dsRNAs, termed “small activating RNAs” (saRNAs). It is currently not known whether RNAa is conserved in other organisms.

Small double-stranded RNA (dsRNA), such as small interfering RNA (siRNA) and microRNA (miRNA), have been found to be the trigger of an evolutionary conserved mechanism known as RNA interference (RNAi). RNAi invariably leads to gene silencing via remodeling chromatin to thereby suppress transcription, degrading complementary mRNA, or blocking protein translation. However, in instances described in detail in the examples section which follows, oligonucleotides are shown to increase the expression and/or function of the Glial cell derived neurotrophic factor (GDNF) polynucleotides and encoded products thereof, dsRNAs may also act as small activating RNAs (saRNA). Without wishing to be bound by theory, by targeting sequences in gene promoters, saRNAs would induce target gene expression in a phenomenon referred to as dsRNA-induced transcriptional activation (RNAa).

In a further embodiment, the “preferred target segments” identified herein may be employed in a screen for additional compounds that modulate the expression of Glial cell derived neurotrophic factor (GDNF) polynucleotides. “Modulators” are those compounds that decrease or increase the expression of a nucleic acid molecule encoding Glial cell derived neurotrophic factor (GDNF) and which comprise at least a 5-nucleotide portion that is complementary to a preferred target segment. The screening method comprises the steps of contacting a preferred target segment of a nucleic acid molecule encoding sense or natural antisense polynucleotides of Glial cell derived neurotrophic factor (GDNF) with one or more candidate modulators, and selecting for one or more candidate modulators which decrease or increase the expression of a nucleic acid molecule encoding Glial cell derived neurotrophic factor (GDNF) polynucleotides, e.g. SEQ ID NOS: 5 to 34. Once it is shown that the candidate modulator or modulators are capable of modulating (e.g. either decreasing or increasing) the expression of a nucleic acid molecule encoding Glial cell derived neurotrophic factor (GDNF) polynucleotides, the modulator may then be employed in further investigative studies of the function of Glial cell derived neurotrophic factor (GDNF) polynucleotides, or for use as a research, diagnostic, or therapeutic agent in accordance with the present invention.

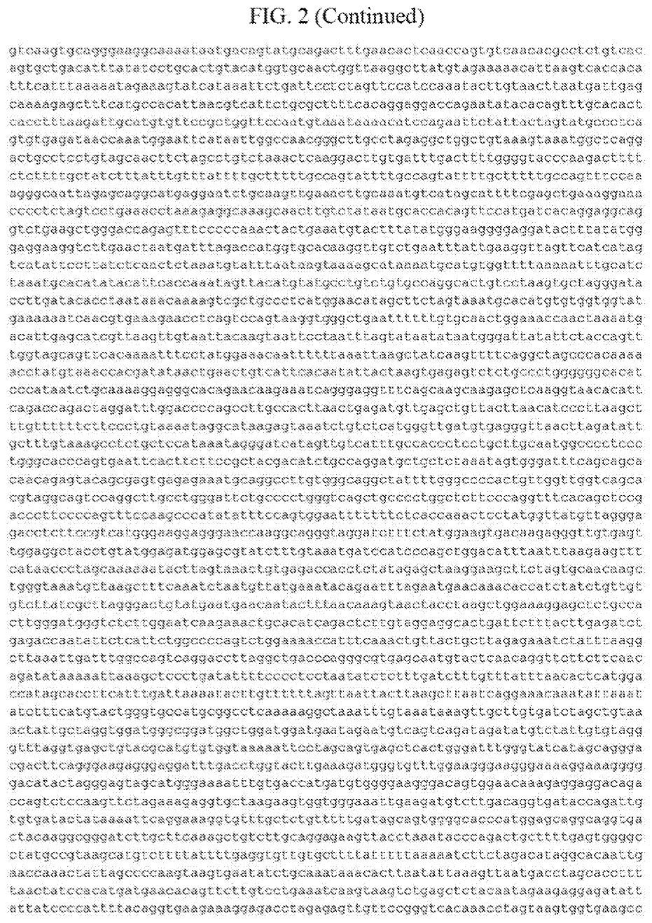

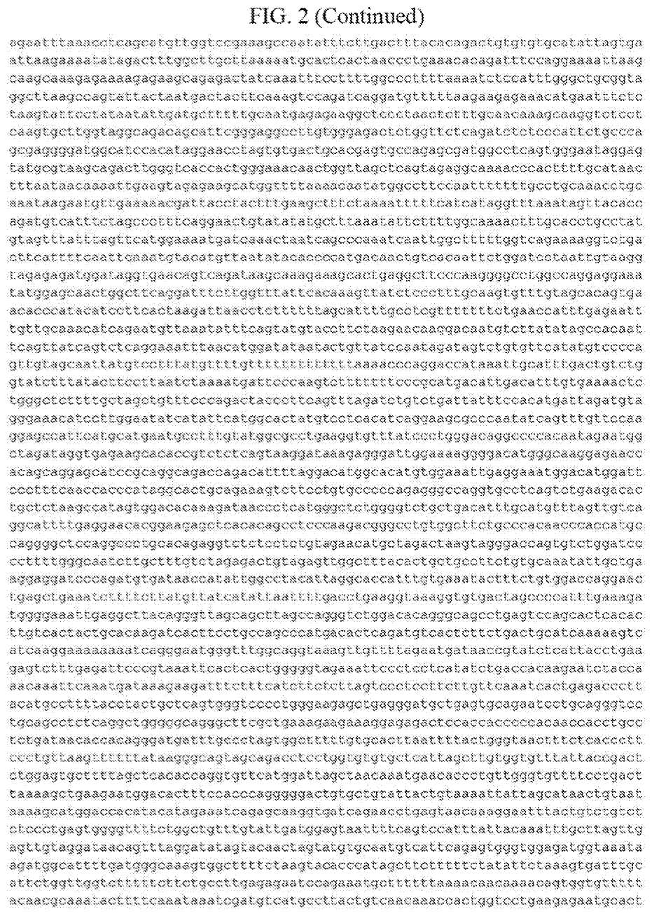

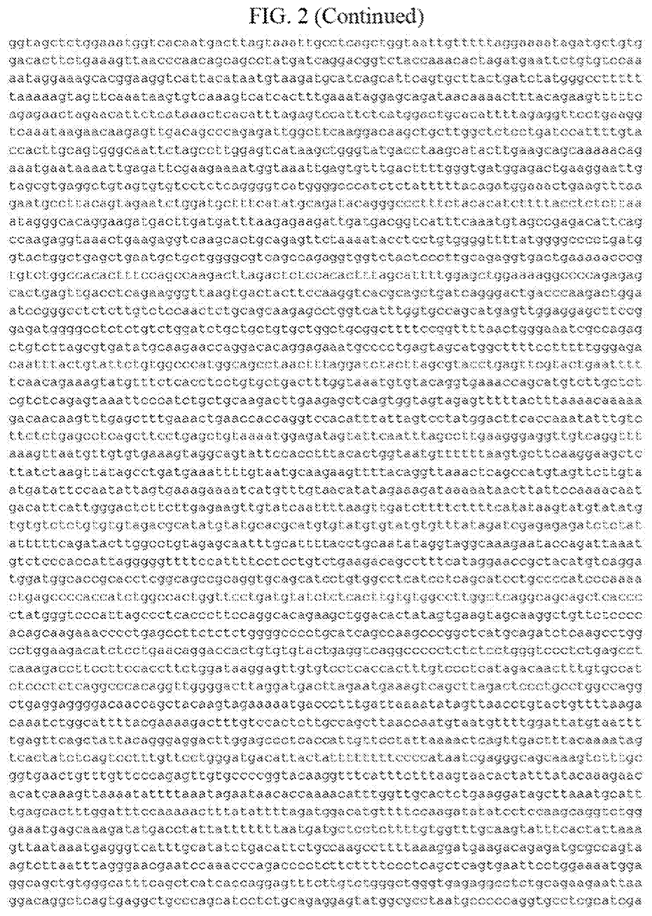

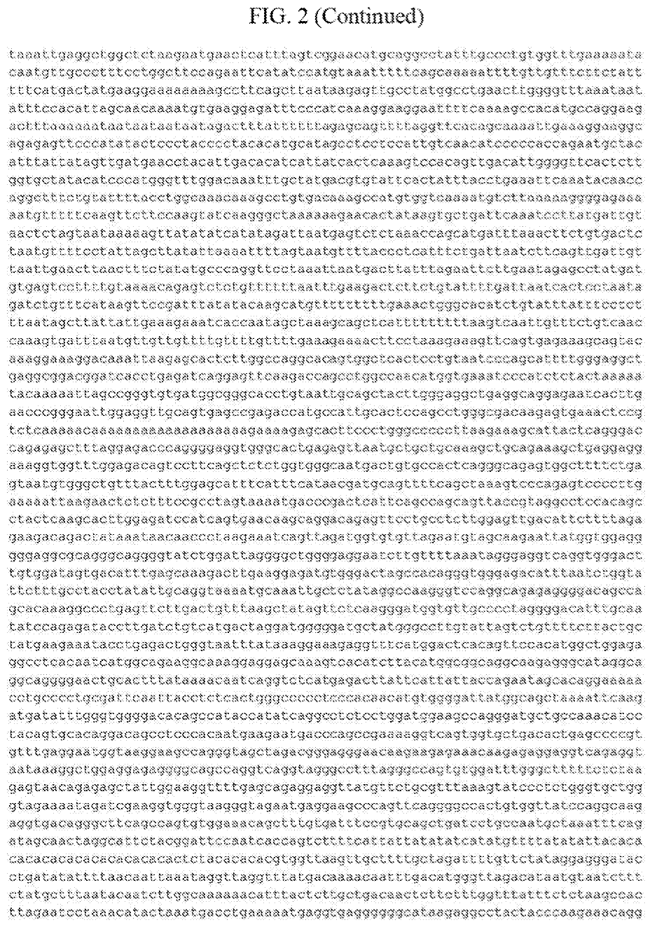

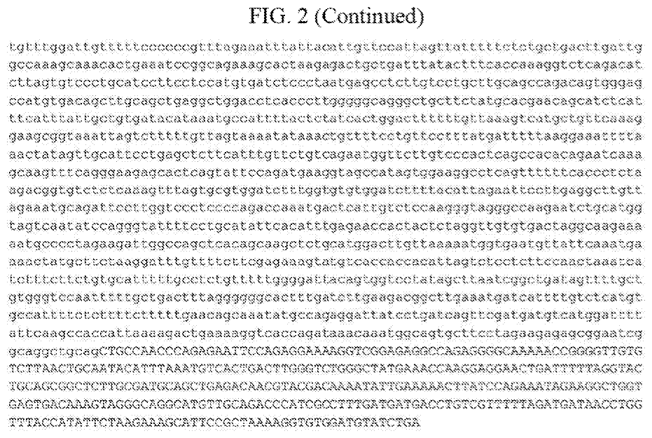

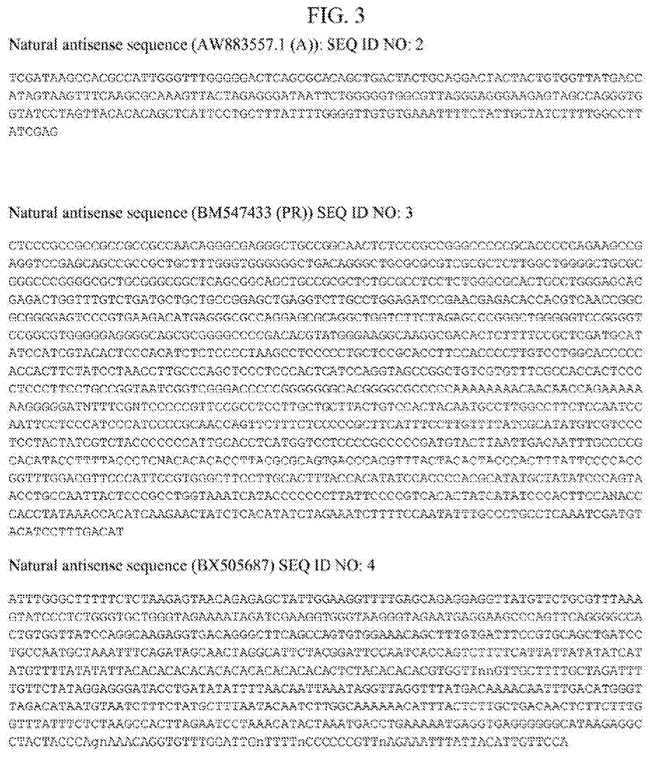

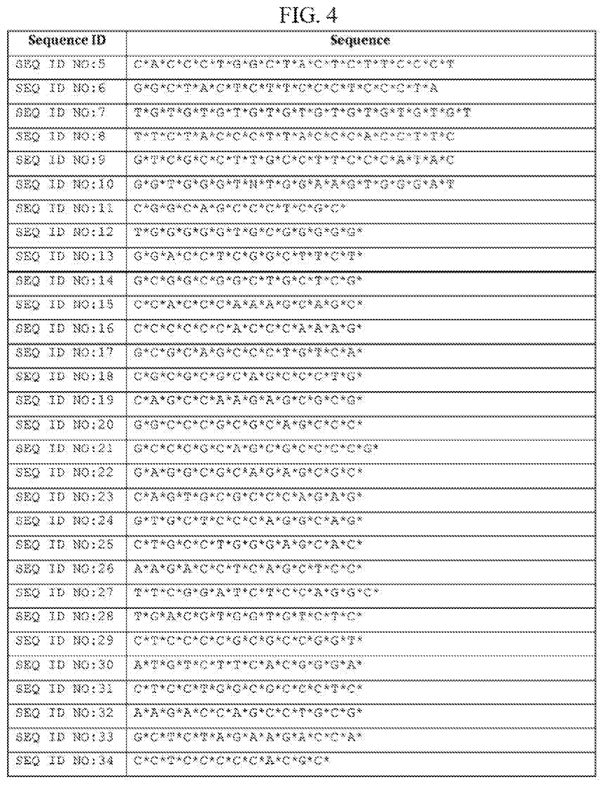

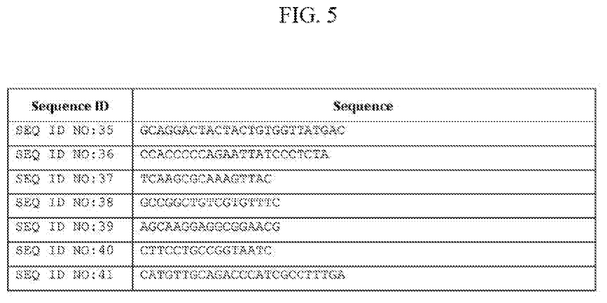

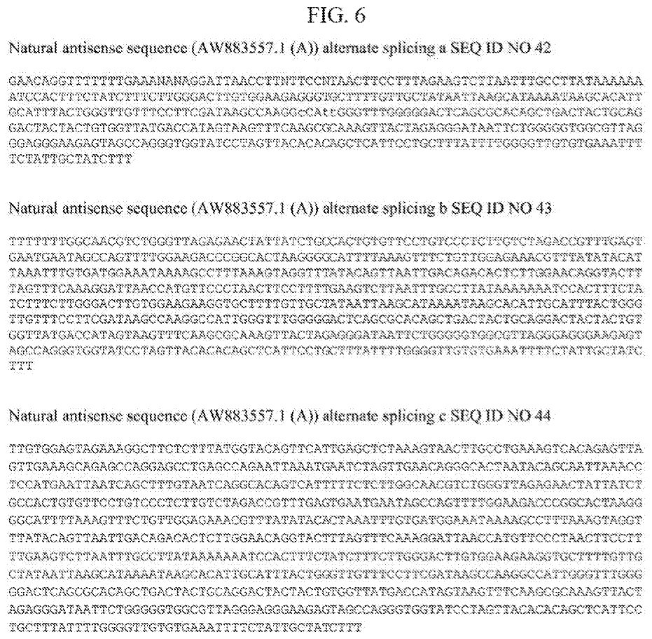

Targeting the natural antisense sequence preferably modulates the function of the target gene. For example, the GDNF gene (NM_199234.1, FIG. 2). In a preferred embodiment, the target is an antisense polynucleotide of the Glial cell derived neurotrophic factor gene. In a preferred embodiment, an antisense oligonucleotide targets sense and/or natural antisense sequences of Glial cell derived neurotrophic factor (GDNF) polynucleotides (NM_199234.1, FIG. 2), variants, alleles, isoforms, homologs, mutants, derivatives, fragments and complementary sequences thereto. Preferably the oligonucleotide is an antisense molecule and the targets include coding and noncoding regions of antisense and/or sense GDNF polynucleotides.

The preferred target segments of the present invention may be also be combined with their respective complementary antisense compounds of the present invention to form stabilized double-stranded (duplexed) oligonucleotides.

Such double stranded oligonucleotide moieties have been shown in the art to modulate target expression and regulate translation as well as RNA processing via an antisense mechanism. Moreover, the double-stranded moieties may be subject to chemical modifications (Fire et al., (1998) Nature, 391, 806-811; Timmons and Fire, (1998) Nature, 395, 854; Timmons et al., (2001) Gene, 263, 103-112; Tabara et al., (1998) Science, 282, 430-431; Montgomery et al., (1998) Proc. Natl. Acad. Sci. USA. 95, 15502-15507; Tuschl et al., (1999) Gene Dev., 13, 3191-3197; Elbashir et al. (2001) Nature, 411, 494-498; Elbashir et al., (2001) Genes Dev. 15, 188-200)). For example, such double-stranded moieties have been shown to inhibit the target by the classical hybridization of antisense strand of the duplex to the target, thereby triggering enzymatic degradation of the target (Tijsterman et al., (2002) Science, 295, 694-697).

In a preferred embodiment, an antisense oligonucleotide targets Glial cell derived neurotrophic factor (GDNF) polynucleotides (e.g. accession number NM_199234.1), variants, alleles, isoforms, homologs, mutants, derivatives, fragments and complementary sequences thereto. Preferably the oligonucleotide is an antisense molecule.

In accordance with embodiments of the invention, the target nucleic acid molecule is not limited to Glial cell derived neurotrophic factor (GDNF) alone but extends to any of the isoforms, receptors, homologs and the like of Glial cell derived neurotrophic factor (GDNF) molecules.

In another preferred embodiment, an oligonucleotide targets a natural antisense sequence of GDNF polynucleotides, for example, polynucleotides set forth as SEQ ID NOS: 2 to 4 and 42 to 44, and any variants, alleles, homologs, mutants, derivatives, fragments and complementary sequences thereto. Examples of antisense oligonucleotides are set forth as SEQ ID NOS: 5 to 34.

In one embodiment, the oligonucleotides are complementary to or bind to nucleic acid sequences of Glial cell derived neurotrophic factor (GDNF) antisense, including without limitation noncoding sense and/or antisense sequences associated with Glial cell derived neurotrophic factor (GDNF) polynucleotides and modulate expression and/or function of Glial cell derived neurotrophic factor (GDNF) molecules.

In another preferred embodiment, the oligonucleotides are complementary to or bind to nucleic acid sequences of GDNF natural antisense, set forth as SEQ ID NO: 2 to 4 and 42 to 44, and modulate expression and/or function of GDNF molecules.

In a preferred embodiment, oligonucleotides comprise sequences of at least 5 consecutive nucleotides of SEQ ID NOS: 5 to 34 and modulate expression and/or function of Glial cell derived neurotrophic factor (GDNF) molecules.

The polynucleotide targets comprise GDNF, including family members thereof, variants of GDNF; mutants of GDNF, including SNPs; noncoding sequences of GDNF; alleles of GDNF; species variants, fragments and the like. Preferably the oligonucleotide is an antisense molecule.

In another preferred embodiment, the oligonucleotide targeting Glial cell derived neurotrophic factor (GDNF) polynucleotides, comprise: antisense RNA, interference RNA (RNAi), short interfering RNA (siRNA); micro interfering RNA (miRNA); a small, temporal RNA (stRNA), or a short, hairpin RNA (shRNA); small RNA-induced gene activation (RNAa); or, small activating RNA (saRNA).

In another preferred embodiment, targeting of Glial cell derived neurotrophic factor (GDNF) polynucleotides, e.g. SEQ ID NOS: 2 to 4 and 42 to 44, modulates the expression or function of these targets. In one embodiment, expression or function is up-regulated as compared to a control. In another preferred embodiment, expression or function is down-regulated as compared to a control.

In another preferred embodiment, antisense compounds comprise sequences set forth as SEQ ID NOS: 5 to 34. These oligonucleotides can comprise one or more modified nucleotides, shorter or longer fragments, modified bonds and the like.

In another preferred embodiment, SEQ ID NOS: 5 to 34 comprise one or more LNA nucleotides.

The modulation of a desired target nucleic acid can be carried out in several ways known in the art. For example, antisense oligonucleotides, siRNA etc. Enzymatic nucleic acid molecules e.g., ribozymes) are nucleic acid molecules capable of catalyzing one or more of a variety of reactions, including the ability to repeatedly cleave other separate nucleic acid molecules in a nucleotide base sequence-specific manner. Such enzymatic nucleic acid molecules can be used, for example, to target virtually any RNA transcript (Zaug et al., 324, Nature 429 1986; Cecil, 260 JAMA 3030, 1988; and Jefferies et al., 17 Nucleic Acids Research 1371, 1989).

Because of their sequence-specificity, trans-cleaving enzymatic nucleic acid molecules show promise as therapeutic agents for human disease (Usman & McSwiggen, (1995) Ann. Rep. Med. Chem. 30, 285-294; Christoffersen and Marr, (1995) J. Med. Chem. 38, 2023-2037). Enzymatic nucleic acid molecules can be designed to cleave specific RNA targets within the background of cellular RNA. Such a cleavage event renders the mRNA non-functional and abrogates protein expression from that RNA. In this manner, synthesis of a protein associated with a disease state can be selectively inhibited.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, (1979) Proc. R. Soc. London, B 205, 435) have been used to evolve new nucleic acid catalysts capable of catalyzing a variety of reactions, such as cleavage and ligation of phosphodiester linkages and amide linkages, (Joyce, (1989) Gene, 82, 83-87; Beaudry et al., (1992) Science 257, 635-641; Joyce, (1992) Scientific American 267, 90-97; Breaker et al., (1994) TIBTECH 12, 268; Bartel et al., (1993) Science 261:1411-1418; Szostak, (1993) TIBS 17, 89-93; Kumar et al., (1995) FASEB J., 9, 1183; Breaker, (1996) Curr. Op. Biotech., 7, 442).

The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min-l in the presence of saturating (10 mM) concentrations of Mg2+ cofactor. An artificial “RNA ligase” ribozyme has been shown to catalyze the corresponding self-modification reaction with a rate of about 100 min-l. In addition, it is known that certain modified hammerhead ribozymes that have substrate binding arms made of DNA catalyze RNA cleavage with multiple turn-over rates that approach 100 min-l. Finally, replacement of a specific residue within the catalytic core of the hammerhead with certain nucleotide analogues gives modified ribozymes that show as much as a 10-fold improvement in catalytic rate. These findings demonstrate that ribozymes can promote chemical transformations with catalytic rates that are significantly greater than those displayed in vitro by most natural self-cleaving ribozymes. It is then possible that the structures of certain selfcleaving ribozymes may be optimized to give maximal catalytic activity, or that entirely new RNA motifs can be made that display significantly faster rates for RNA phosphodiester cleavage.

Intermolecular cleavage of an RNA substrate by an RNA catalyst that fits the “hammerhead” model was first shown in 1987 (Uhlenbeck, O. C. (1987) Nature, 328: 596-600). The RNA catalyst was recovered and reacted with multiple RNA molecules, demonstrating that it was truly catalytic.

Catalytic RNAs designed based on the “hammerhead” motif have been used to cleave specific target sequences by making appropriate base changes in the catalytic RNA to maintain necessary base pairing with the target sequences (Haseloff and Gerlach, (1988) Nature, 334, 585; Walbot and Bruening, (1988) Nature, 334, 196; Uhlenbeck, O. C. (1987) Nature, 328; 596-600; Korman, et al. (1988) FEBS Lett., 228: 228-230). This has allowed use of the catalytic RNA to cleave specific target sequences and indicates that catalytic RNAs designed according to the “hammerhead” model may possibly cleave specific substrate RNAs in vivo. (see Haseloff and Gerlach, (1988) Nature, 334, 585; Walbot and Bruening, (1988) Nature, 334, 196; Uhlenbeck, O. C (1987) Nature, 328:596-600).

RNA interference (RNAi) has become a powerful tool for modulating gene expression in mammals and mammalian cells. This approach requires the delivery of small interfering RNA (siRNA) either as RNA itself or as DNA, using an expression plasmid or virus and the coding sequence for small hairpin RNAs that are processed to siRNAs. This system enables efficient transport of the pre-siRNAs to the cytoplasm where they are active and permit the use of regulated and tissue specific promoters for gene expression.

In a preferred embodiment, an oligonucleotide or antisense compound comprises an oligomer or polymer of ribonucleic acid (RNA) and/or deoxyribonucleic acid (DNA), or a mimetic, chimera, analog or homolog thereof. This term includes oligonucleotides composed of naturally occurring nucleotides, sugars and covalent internucleoside (backbone) linkages as well as oligonucleotides having non-naturally occurring portions which function similarly. Such modified or substituted oligonucleotides are often desired over native forms because of desirable properties such as, for example, enhanced cellular uptake, enhanced affinity for a target nucleic acid and increased stability in the presence of nucleases.

According to the present invention, the oligonucleotides or “antisense compounds” include antisense oligonucleotides (e.g. RNA, DNA, mimetic, chimera, analog or homolog thereof), ribozymes, external guide sequence (EGS) oligonucleotides, siRNA compounds, single- or double-stranded RNA interference (RNAi) compounds such as siRNA compounds, saRNA, aRNA, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, double-stranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleotide positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines (Hammond et al., (1991) Nat. Rev. Genet., 2, 110-119; Matzke et at, (2001) Curr. Opin. Genet Dev., 11, 221-227; Sharp, (2001) Genes Dev., 15, 485-490). When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

Once introduced to a system, the compounds of the invention may elicit the action of one or more enzymes or structural proteins to effect cleavage or other modification of the target nucleic acid or may work via occupancy-based mechanisms. In general, nucleic acids (including oligonucleotides) may be described as “DNA-like” (i.e., generally having one or more 2′-deoxy sugars and, generally, T rather than U bases) or “RNA-like” (i.e., generally having one or more 2′-hydroxyl or 2′-modified sugars and, generally U rather than T bases). Nucleic acid helices can adopt more than one type of structure, most commonly the A- and B-forms. It is believed that, in general, oligonucleotides which have B-form-like structure are “DNA-like” and those which have A-formlike structure are “RNA-like.” In some (chimeric) embodiments, an antisense compound may contain both A- and B-form regions.

The antisense compounds in accordance with this invention can comprise an antisense portion from about 5 to about 80 nucleotides (i.e. from about 5 to about 80 linked nucleosides) in length. This refers to the length of the antisense strand or portion of the antisense compound. In other words, a single-stranded antisense compound of the invention comprises from 5 to about 80 nucleotides, and a double-stranded antisense compound of the invention (such as a dsRNA, for example) comprises a sense and an antisense strand or portion of S to about 80 nucleotides in length. One of ordinary skill in the art will appreciate that this comprehends antisense portions of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range therewithin.

In one embodiment, the antisense compounds of the invention have antisense portions of 10 to 50 nucleotides in length. One having ordinary skill in the art will appreciate that this embodies oligonucleotides having antisense portions of 10, II, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the oligonucleotides are 15 nucleotides in length.

In one embodiment, the antisense or oligonucleotide compounds of the invention have antisense portions of 12 or 13 to 30 nucleotides in length. One having ordinary skill in the art will appreciate that this embodies antisense compounds having antisense portions of 12, 13, 14, 15, 16, 17, 18, 19, 20, 21, 22, 23, 24, 25, 26, 27, 28, 29 or 30 nucleotides in length, or any range there within.

What is claimed is:

1. A method of upregulating a function of and/or the expression of a Glial cell derived neurotrophic factor (GDNF) polynucleotide having SEQ ID NO: 1 in patient cells or tissues in vivo or in vitro comprising:
contacting said cells or tissues with at least one single stranded antisense oligonucleotide of 14 to 30 nucleotides in length that targets and specifically hybridizes and is at least 95% complementary to a complementary region of a polynucleotide selected from SEQ ID NO: 4, thereby upregulating a function of and/or the expression of the Glial cell derived neurotrophic factor (GDNF) polynucleotide in patient cells or tissues in vivo or in vitro.

2. The method of claim 1, wherein a function of and/or the expression of the Glial cell derived neurotrophic factor (GDNF) is increased in vivo or in vitro with respect to a control.

3. The method of claim 1, wherein the at least one antisense oligonucleotide comprises one or more modifications selected from: at least one modified sugar moiety, at least one modified internucleoside linkage, at least one modified nucleotide, and combinations thereof.

4. The method of claim 3, wherein the one or more modifications comprise at least one modified sugar moiety selected from: a 2′-O-methoxyethyl modified sugar moiety, a 2′-methoxy modified sugar moiety, a 2′-O-alkyl modified sugar moiety, a bicyclic sugar moiety, and combinations thereof.

5. The method of claim 3, wherein the one or more modifications comprise at least one modified internucleoside linkage selected from: a phosphorothioate, alkylphosphonate, phosphorodithioate, alkylphosphonothioate, phosphoramidate, carbamate, carbonate, phosphate triester, acetamidate, carboxymethyl ester, and combinations thereof.

6. The method of claim 3, wherein the one or more modifications comprise at least one modified nucleotide selected from: a peptide nucleic acid (PNA), a locked nucleic acid (LNA), an arabino-nucleic acid (FANA), an analogue, a derivative, and combinations thereof.

7. The method of claim 1, wherein the at least one oligonucleotide comprises at least one of the oligonucleotide sequences set forth as SEQ ID NOS: 16, 18, 19, 22, 23, 26, 33 and 34.

8. A method of upregulating a function of and/or the expression of a Glial cell derived neurotrophic factor (GDNF) polynucleotide having SEQ ID NO: 1 in patient cells or tissues in vivo or in vitro comprising:
contacting said cells or tissues with at least one single stranded antisense oligonucleotide of 10 to 30 nucleotides in length that targets and specifically hybridizes to a complementary region of a polynucleotide selected from the group consisting of SEQ ID NO: 3;
thereby upregulating a function of and/or the expression of the Glial cell derived neurotrophic factor (GDNF) polynucleotide in patient cells or tissues in vivo or in vitro.

9. The method of claim 8, wherein a function of and/or the expression of the Glial cell derived neurotrophic factor (GDNF) is increased in vivo or in vitro with respect to a control.

10. The method of claim 8, wherein the at least one antisense oligonucleotide comprises one or more modifications selected from: at least one modified sugar moiety, at least one modified internucleoside linkage, at least one modified nucleotide, and combinations thereof.

11. The method of claim 8, wherein the one or more modifications comprise at least one modified sugar moiety selected from: a 2′-O-methoxyethyl modified sugar moiety, a 2′-methoxy modified sugar moiety, a 2′-O-alkyl modified sugar moiety, a bicyclic sugar moiety, and combinations thereof.

12. The method of claim 8, wherein the one or more modifications comprise at least one modified internucleoside linkage selected from: a phosphorothioate, alkylphosphonate, phosphorodithioate, alkylphosphonothioate, phosphoramidate, carbamate, carbonate, phosphate triester, acetamidate, carboxymethyl ester, and combinations thereof.

13. The method of claim 8, wherein the one or more modifications comprise at least one modified nucleotide selected from: a peptide nucleic acid (PNA), a locked nucleic acid (LNA), an arabino-nucleic acid (FANA), an analogue, a derivative, and combinations thereof.

14. The method of claim 8, wherein the at least one oligonucleotide comprises at least one of the oligonucleotide sequences set forth as SEQ ID NOS: 8, 9 and 10.

15. A method of upregulating a function of and/or the expression of a Glial cell derived neurotrophic factor (GDNF) polynucleotide having SEQ ID NO: 1 in patient cells or tissues in vivo or in vitro comprising:
contacting said cells or tissues with at least one single stranded antisense oligonucleotide of 10 to 30 nucleotides in length that targets and specifically hybridizes to a complementary region of a polynucleotide selected from the group consisting of AW883557.1 and splice variants thereof;
thereby upregulating a function of and/or the expression of the Glial cell derived neurotrophic factor (GDNF) polynucleotide in patient cells or tissues in vivo or in vitro.

16. The method of claim 15, wherein a function of and/or the expression of the Glial cell derived neurotrophic factor (GDNF) is increased in vivo, or in vitro with respect to a control.

17. The method of claim 15, wherein the at least one antisense oligonucleotide comprises one or more modifications selected from: at least one modified sugar moiety, at least one modified internucleoside linkage, at least one modified nucleotide, and combinations thereof.

18. The method of claim 15, wherein the one or more modifications comprise at least one modified sugar moiety selected from: a 2′-O-methoxyethyl modified sugar moiety, a 2′-methoxy modified sugar moiety, a 2′-O-alkyl modified sugar moiety, a bicyclic sugar moiety, and combinations thereof.

19. The method of claim 15, wherein the one or more modifications comprise at least one modified internucleoside linkage selected from: a phosphorothioate, alkylphosphonate, phosphorodithioate, alkylphosphonothioate, phosphoramidate, carbamate, carbonate, phosphate triester, acetamidate, carboxymethyl ester, and combinations thereof.

20. The method of claim 15, wherein the one or more modifications comprise at least one modified nucleotide selected from: a peptide nucleic acid (PNA), a locked nucleic acid (LNA), an arabino-nucleic acid (FANA), an analogue, a derivative, and combinations thereof.

21. The method of claim 15, wherein the at least one oligonucleotide comprises at least one of the oligonucleotide sequence set forth as SEQ ID NO: 5.

22. A method of upregulating a function of and/or the expression of a Glial cell derived neurotrophic factor (GDNF) gene in mammalian cells or tissues in vivo or in vitro comprising:
contacting said cells or tissues with at least one short interfering RNA (siRNA) oligonucleotide of about 19 to about 30 nucleotides in length, said at least one siRNA oligonucleotide specifically hybridizing to a non-overlapping region of a natural antisense polynucleotide of a Glial cell derived neurotrophic factor (GDNF) polynucleotide, wherein said at least one siRNA oligonucleotide has at least 95% sequence complementarity to a said natural antisense polynucleotide of the Glial cell derived neurotrophic factor (GDNF) polynucleotide;
and upregulating a function of and/or the expression of Glial cell derived neurotrophic factor (GDNF) in mammalian cells or tissues in vivo or in vitro.

23. A synthetic, modified single stranded oligonucleotide comprising at least one modification wherein the at least one modification is selected from: at least one modified sugar moiety; at least one modified internucleotide linkage; at least one modified nucleotide, and combinations thereof; wherein said oligonucleotide is an antisense compound of 14-30 nucleotides in length which is 100% complementary to and specifically hybridizes with a natural antisense transcript selected from BX505687 (SEQ ID NO: 4) and upregulates the function and/or expression of a Glial cell derived neurotrophic factor (GDNF) gene in vivo or in vitro as compared to a normal control.

24. The oligonucleotide of claim 23, wherein the at least one modification comprises an internucleotide linkage selected from the group consisting of: phosphorothioate, alkylphosphonate, phosphorodithioate, alkylphosphonothioate, phosphoramidate, carbamate, carbonate, phosphate triester, acetamidate, carboxymethyl ester, and combinations thereof.

25. The oligonucleotide of claim 23, wherein said oligonucleotide comprises at least one phosphorothioate internucleotide linkage.

26. The oligonucleotide of claim 23, wherein said oligonucleotide comprising a backbone of phosphorothioate internucleotide linkages.

27. The oligonucleotide of claim 23, wherein the oligonucleotide comprises at least one modified nucleotide, said modified nucleotide selected from: a peptide nucleic acid, a locked nucleic acid (LNA), analogue, derivative, and a combination thereof.

28. The oligonucleotide of claim 23, wherein the oligonucleotide comprises a plurality of modifications, wherein said modifications comprise modified nucleotides selected from: phosphorothioate, alkylphosphonate, phosphorodithioate, alkylphosphonothioate, phosphoramidate, carbamate, carbonate, phosphate triester, acetamidate, carboxymethyl ester, and a combination thereof.

29. The oligonucleotide of claim 23, wherein the oligonucleotide comprises a plurality of modifications, wherein said modifications comprise modified nucleotides selected from: peptide nucleic acids, locked nucleic acids (LNA), analogues, derivatives, and a combination thereof.

30. The oligonucleotide of claim 23, wherein the oligonucleotide comprises at least one modified sugar moiety selected from: a 2′-O-methoxyethyl modified sugar moiety, a 2′-methoxy modified sugar moiety, a 2′-O-alkyl modified sugar moiety, a bicyclic sugar moiety, and a combination thereof.

31. The oligonucleotide of claim 23, wherein the oligonucleotide comprises a plurality of modifications, wherein said modifications comprise modified sugar moieties selected from: a 2′-O-methoxyethyl modified sugar moiety, a 2′-methoxy modified sugar moiety, a 2′-O-alkyl modified sugar moiety, a bicyclic sugar moiety, and a combination thereof.

32. The oligonucleotide of claim 23, wherein the oligonucleotide comprises SEQ ID NOS: 16, 18, 19, 21, 22, 23, 26, 33 and 34.

33. A synthetic, modified single stranded oligonucleotide comprising at least one modification wherein the at least one modification is selected from: at least one modified sugar moiety; at least one modified internucleotide linkage; at least one modified nucleotide, and combinations thereof; wherein said oligonucleotide is an antisense compound of 10-30 nucleotides in length which is at least 95% complementary to and specifically hybridizes with a polynucleotide selected from the group consisting of SEQ ID NO: 3 and upregulates the function and/or expression of a Glial cell derived neurotrophic factor (GDNF) gene in vivo or in vitro as compared to a normal control.

34. The oligonucleotide of claim 33, wherein the at least one modification comprises an internucleotide linkage selected from the group consisting of: phosphorothioate, alkylphosphonate, phosphorodithioate, alkylphosphonothioate, phosphoramidate, carbamate, carbonate, phosphate triester, acetamidate, carboxymethyl ester, and combinations thereof.

35. The oligonucleotide of claim 33, wherein said oligonucleotide comprises at least one phosphorothioate internucleotide linkage.

36. The oligonucleotide of claim 33, wherein said oligonucleotide comprising a backbone of phosphorothioate internucleotide linkages.

37. The oligonucleotide of claim 33, wherein the oligonucleotide comprises at least one modified nucleotide, said modified nucleotide selected from: a peptide nucleic acid, a locked nucleic acid (LNA), analogue, derivative, and a combination thereof.

38. The oligonucleotide of claim 33, wherein the oligonucleotide comprises a plurality of modifications, wherein said modifications comprise modified nucleotides selected from: phosphorothioate, alkylphosphonate, phosphorodithioate, alkylphosphonothioate, phosphoramidate, carbamate, carbonate, phosphate triester, acetamidate, carboxymethyl ester, and a combination thereof.

39. The oligonucleotide of claim 33, wherein the oligonucleotide comprises a plurality of modifications, wherein said modifications comprise modified nucleotides selected from: peptide nucleic acids, locked nucleic acids (LNA), analogues, derivatives, and a combination thereof.

40. The oligonucleotide of claim 33, wherein the oligonucleotide comprises at least one modified sugar moiety selected from: a 2′-O-methoxyethyl modified sugar moiety, a 2′-methoxy modified sugar moiety, a 2′-O-alkyl modified sugar moiety, a bicyclic sugar moiety, and a combination thereof.

41. The oligonucleotide of claim 33, wherein the oligonucleotide comprises a plurality of modifications, wherein said modifications comprise modified sugar moieties selected from: a 2′-O-methoxyethyl modified sugar moiety, a 2′-methoxy modified sugar moiety, a 2′-O-alkyl modified sugar moiety, a bicyclic sugar moiety, and a combination thereof.

42. The oligonucleotide of claim 34, wherein the oligonucleotide comprises SEQ ID NOS: 8, 9 and 10.

43. A synthetic, modified single stranded oligonucleotide comprising at least one modification wherein the at least one modification is selected from: at least one modified sugar moiety: at least one modified internucleotide linkage; at least one modified nucleotide, and combinations thereof; wherein said oligonucleotide is an antisense compound of 10-30 nucleotides in length which is at least 95% complementary to and specifically hybridizes with a polynucleotide selected from the group consisting of AW883557.1 and splice variants thereof and upregulates the function and/or expression of a Glial cell derived neurotrophic factor (GDNF) gene in viva or in vitro as compared to a normal control.

44. The oligonucleotide according to claim 43 selected from SEQ ID NO: 5.

45. A pharmaceutical composition comprising one or more oligonucleotides according to claim 23 and a pharmaceutically acceptable excipient.

46. A pharmaceutical composition comprising one or more oligonucleotides according to claim 33 and a pharmaceutically acceptable excipient.

47. A pharmaceutical composition comprising one or more oligonucleotides according to claim 43 and a pharmaceutically acceptable excipient.

48. A modified oligonucleotide selected from the group consisting of SEQ ID NOS: 5, 9, 10, 16, 18, 19, 21, 22, 26, 33 or 34.

49. A method of treating a disease associated with at least one Glial cell derived neurotrophic factor (GDNF) polynucleotide and/or at least one encoded product thereof selected from a neurological disease or disorder, comprising:
administering to a patient a therapeutically effective dose of at least one antisense oligonucleotide of 10 to 30 nucleotides in length that binds and specifically hybridizes to a natural antisense polynucleotide of said at least one Glial cell derived neurotrophic factor (GDNF) polynucleotide having SEQ ID NO: 4 and upregulates expression of said at least one Glial cell derived neurotrophic factor (GDNF) polynucleotide;
thereby treating the disease associated with the at least one Glial cell derived neurotrophic factor (GDNF) polynucleotide and/or at least one encoded product thereof.

50. The method of claim 49, wherein the neurological disease or disorder is selected from: a disease or a disorder associated with defective neurogenesis; a neurodegenerative disease or disorder selected from Alzheimer's disease, Parkinson's disease, Huntington's disease, amyotrophic lateral sclerosis; a neuropsychiatric disorder (depression, schizophrenia, schizofreniform disorder, schizoaffective disorder, and delusional disorder; anxiety disorders such as panic disorder, phobias (including agoraphobia), an obsessive-compulsive disorder, a posttraumatic stress disorder, a bipolar disorder, anorexia nervosa, bulimia nervosa, an autoimmune disorder of the central nervous system (multiple sclerosis), memory loss, a long term or a short term memory disorder, benign forgetfulness, a childhood learning disorder, close head injury, an attention deficit disorder, neuronal reaction to viral infection, brain damage, narcolepsy, a sleep disorder selected from circadian rhythm disorders, insomnia and narcolepsy; severance of nerves or nerve damage, severance of cerebrospinal nerve cord and a damage to brain or nerve cells, a neurological deficit associated with AIDS, a motor and tic disorder characterized by motor and/or vocal tics selected from Tourette's disorder, chronic motor or vocal tic disorder, transient tic disorder, and stereotypic movement disorder, a substance abuse disorder selected from substance dependence, substance abuse and the sequalae of substance abuse/dependence, such as substance-induced psychological disorder, substance withdrawal and substance-induced dementia or amnestic disorder), traumatic brain injury, tinnitus, neuralgia, trigeminal neuralgia, pain, chronic pain, chronic inflammatory pain, pain associated with arthritis, fibromyalgia, back pain, cancer-associated pain, pain associated with digestive disease, pain associated with Crohn's disease, pain associated with autoimmune disease, pain associated with endocrine disease, pain associated with diabetic neuropathy, phantom limb pain, spontaneous pain, chronic post-surgical pain, chronic temporomandibular pain, causalgia, post-herpetic neuralgia, AIDS-related pain, complex regional pain syndromes type I and II, trigeminal neuralgia, chronic back pain, pain associated with spinal cord injury, pain associated with drug intake and recurrent acute pain, neuropathic pain, inappropriate neuronal activity resulting in neurodysthesias in a disease such as diabetes, an MS and a motor neuron disease, ataxias, muscular rigidity, spasticity, temporomandibular joint dysfunction, Reward deficiency syndrome (RDS), neurotoxicity caused by alcohol or substance abuse selected from ecstacy, methamphetamine or other drugs of abuse, mental retardation or cognitive impairment selected from nonsyndromic X-linked mental retardation, fragile X syndrome, Down's syndrome, autism), aphasia, Bell's palsy, Creutzfeldt-jacob disease, encephalitis, age related macular degeneration, ondine syndrome, WAGR syndrome, hearing loss, Werdnig-Hoffmann disease, chronic proximal spinal muscular atrophy, Guillain-Barre syndrome, Multiple System Atrophy, Shy Drager Syndrome, Rett syndrome, epilepsy, spinal cord injury, stroke, hypoxia, ischemia, brain injury, diabetic neuropathy, peripheral neuropathy, nerve transplantation complications, motor neuron disease, peripheral nerve injury.

51. A method of treating a disease associated with at least one Glial cell derived neurotrophic factor (GDNF) polynucleotide and/or at least one encoded product thereof selected from a neurological disease or disorder, comprising:
administering to a patient a therapeutically effective dose of at least one antisense oligonucleotide of 10 to 30 nucleotides in length that binds and specifically hybridizes to a natural antisense polynucleotide of said at least one Glial cell derived neurotrophic factor (GDNF) polynucleotide having SEQ ID NO: 3 and upregulates expression of said at least one Glial cell derived neurotrophic factor (GDNF) polynucleotide;
thereby treating the disease associated with the at least one Glial cell derived neurotrophic factor (GDNF) polynucleotide and/or at least one encoded product thereof.

52. A method of treating a disease associated with at least one Glial cell derived neurotrophic factor (GDNF) polynucleotide and/or at least one encoded product thereof selected from a neurological disease or disorder, comprising:
administering to a patient a therapeutically effective dose of at least one antisense oligonucleotide of 10 to 30 nucleotides in length that binds and specifically hybridizes to a natural antisense polynucleotide of said at least one Glial cell derived neurotrophic factor (GDNF) polynucleotide selected from AW883557.1 or a splice variant thereof and upregulates expression of said at least one Glial cell derived neurotrophic factor (GDNF) polynucleotide;
thereby treating the disease associated with the at least one Glial cell derived neurotrophic factor (GDNF) polynucleotide and/or at least one encoded product thereof.
